(19) United States
(12) Patent Application Publication
(10) Pub. No.: US 2025/0368668 A1
(43) Pub. Date: Dec. 4, 2025

(54) CRYSTALLINE FORMS AND SALT FORMS OF A CARDIAC P2X RECEPTOR AGONIST, PHARMACEUTICAL COMPOSITIONS, AND THEIR USE IN TREATING MEDICAL DISORDERS

(71) Applicant: Cornovus Pharmaceuticals, Inc., New Haven, CT (US)

(72) Inventors: David T. Jonaitis, Brookston, IN (US); Lisa Michelle Grove, Lafayette, IN (US); Scott S. Harried, Sun Prairie, WI (US)

(21) Appl. No.: 19/226,237

(22) Filed: Jun. 3, 2025

Related U.S. Application Data
(60) Provisional application No. 63/655,312, filed on Jun. 3, 2024.

(51) Int. Cl. C07F 9/6561; A61K 31/675

(52) U.S. Cl. CPC C07F 9/65616; A61K 31/675; C07B 2200/13

(57) ABSTRACT

The invention provides crystalline hydrate forms and a sodium salt of a purinyl-dihydroxybicyclo[3.1.0]hexanyl phosphate compound, pharmaceutical compositions, their use in the treatment of a disease or condition, such as heart failure, and methods for making pharmaceutical compositions.

### CROSS-REFERENCE TO RELATED APPLICATIONS

This application claims the benefit of and priority to U.S. Provisional Patent Application Ser. No. 63/655,312, filed Jun. 3, 2024, the contents of which are hereby incorporated by reference in their entirety.

### FIELD OF THE INVENTION

The invention provides crystalline hydrate forms and a sodium salt of a purinyl-dihydroxybicyclo[3.1.0]hexanyl phosphate compound, pharmaceutical compositions, their use in the treatment of a disease or condition, such as heart failure, and methods for making pharmaceutical compositions.

### BACKGROUND

Heart failure affects a substantial number of patients worldwide and is characterized by impairment of the heart's ability to pump blood. Exemplary symptoms of heart failure include, for example, shortness of breath, excessive fatigue, and leg swelling. Coronary artery disease, heart attack, high blood pressure, atrial fibrillation, valvular heart disease, excessive alcohol consumption, infection, and cardiomyopathy are factors that increase a patient's risk for experiencing heart failure.

Stimulation of cardiac P2X receptors has been reported to provide benefits to subjects suffering from heart failure. See, for example, Zhou et al. in J. Pharmacol. Exp. Ther. (2010) vol. 333(3), pages 920-928. P2X receptors are expressed in cardiac tissue, and cardiac P2X receptor stimulation causes an increase of nitric oxide and cyclic guanosine monophosphate (cGMP) in cardiomyocytes. Additional compounds and therapeutic methods using a cardiac P2X receptor agonist are described in U.S. Pat. Nos. 9,303,053 and 9,526,739 and in J. Med. Chem. (2010) vol. 53, pages 2562-2576.

Compounds with superior properties for manufacture and use to treat heart failure that are stable are needed and would benefit patients. The present invention addresses this need and provides other related advantages.

### SUMMARY

The invention provides crystalline hydrate forms and a sodium salt of a purinyl-dihydroxybicyclo[3.1.0]hexanyl phosphate compound, pharmaceutical compositions, their use in the treatment of a disease or condition, such as heart failure, and methods for making pharmaceutical compositions. In particular, one aspect of the invention provides a compound that is crystalline hydrate of compound I:

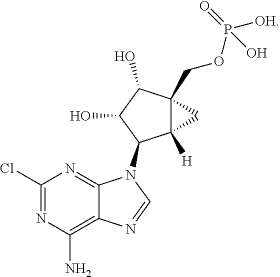 [SMILES: Nc1nc(Cl)nc2c1ncn2[C@H]1[C@H](O)[C@H](O)[C@]2(COP(=O)(O)O)C[C@H]12]

One benefit of the crystalline hydrate of compound I is that it there are fewer crystalline forms than with the crystalline sodium salt of compound I, thereby reducing risk of generating a different crystalline form of the compound during manufacturing procedures. Additionally, Form B of crystalline hydrate of compound I was found to be more stable than Form A of crystalline hydrate of compound I and more stable than the crystalline sodium salt forms of compound I. Further description of additional features of the compounds are described in the detailed description. The compounds may be part of a pharmaceutical composition comprising a pharmaceutically acceptable carrier.

Another aspect of the invention provides a compound that is a sodium salt of compound I:

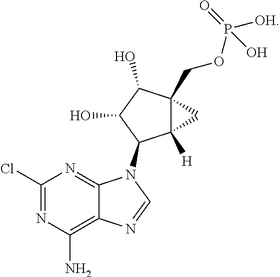 [SMILES: Nc1nc(Cl)nc2c1ncn2[C@H]1[C@H](O)[C@H](O)[C@]2(COP(=O)(O)O)C[C@H]12]

In certain embodiments, the sodium salt of compound I is crystalline. Further description of additional features of the compounds are described in the detailed description. The compounds may be part of a pharmaceutical composition comprising a pharmaceutically acceptable carrier.

Another aspect of the invention provides a method of treating a cardiac disorder, wherein the method comprises administering to a subject in need thereof a therapeutically effective amount of a compound described herein to treat the cardiac disorder.

Another aspect of the invention provides a method for improving cardiac contractile performance in a subject, wherein the method comprises administering to a subject in need thereof an effective amount of an compound described herein to improve cardiac contractile performance.

Another aspect of the invention provides a method for improving cardiac function in a subject, wherein the method comprises administering to a subject in need thereof an effective amount of a compound described herein to improve cardiac function.

Another aspect of the invention provides a method of stimulating the activity of a cardiac P2X receptor in a subject, comprising administering to the subject in need thereof an effective amount of a compound described herein to stimulate the activity of said cardiac P2X receptor.

Another aspect of the invention provides a method of preparing a crystalline hydrate of compound I, wherein the method comprises the steps of:
(a) admixing compound I, methanol, and water to form a mixture, and heating said mixture to a temperature greater than 30° C. to form a heated mixture; and
(b) cooling said heated mixture to thereby form a precipitate that is the crystalline hydrate of compound I;
wherein compound I is represented by:

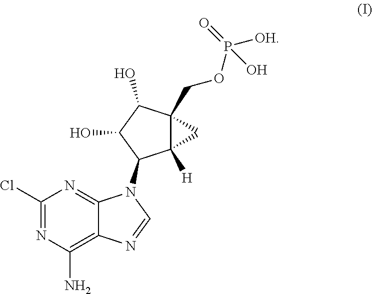 [SMILES: Nc1nc(Cl)nc2c1ncn2[C@H]1[C@H](O)[C@H](O)[C@]2(COP(=O)(O)O)C[C@H]12]

Another aspect of the invention provides a crystalline hydrate of compound I made according to a method described herein.

Another aspect of the invention provides a method of preparing a pharmaceutical composition, wherein the method comprises admixing (i) a crystalline hydrate compound described herein or a salt compound described and (ii) a pharmaceutically acceptable carrier. In certain embodiments, the method comprises admixing (i) a crystalline hydrate compound described herein and (ii) a pharmaceutically acceptable carrier. In certain embodiments, the pharmaceutically acceptable carrier comprises water.

Another aspect of the invention provides a pharmaceutical composition made according to a method described herein.

### BRIEF DESCRIPTION OF THE DRAWINGS

FIG. 1 depicts an X-ray powder diffractogram of Form A crystalline hydrate of compound I, as further described in Example 2.

FIG. 2 depicts a thermogravimetric analysis curve of Form A crystalline hydrate of compound I, as further described in Example 2.

FIG. 3 depicts results of dynamic vapor sorption experiments conducted on Form A crystalline hydrate of compound I, as further described in Example 2.

FIG. 4 depicts an X-ray powder diffractogram of Form B crystalline hydrate of compound I, as further described in Example 5.

FIG. 5 depicts a ¹H NMR spectrum of a sample of Form B crystalline hydrate of compound I that was dissolved in deuterated solvent and then subjected to ¹H NMR analysis, as further described in Example 5.

FIG. 6 depicts an X-ray powder diffractogram of Form A Crystalline Monosodium Salt of Compound I, as further described in Example 6.

FIG. 7 depicts an X-ray powder diffractogram of Form B Crystalline Disodium Salt of Compound I, as further described in Example 7.

FIG. 8 depicts an X-ray powder diffractogram of Form C Crystalline Monosodium Salt of Compound I, as further described in Example 8.

FIG. 9 depicts a differential scanning calorimetry curve and a thermogravimetric analysis curve of Form C Crystalline Monosodium Salt of Compound I, as further described in Example 8.

FIG. 10 depicts results of a dynamic vapor sorption experiment conducted on Form C Crystalline Monosodium Salt of Compound I, as further described in Example 8.

FIG. 11 depicts an X-ray powder diffractogram of Form D Crystalline Monosodium Salt of Compound I, as further described in Example 9.

FIG. 12 depicts a differential scanning calorimetry curve and a thermogravimetric analysis curve of Form D Crystalline Monosodium Salt of Compound I, as further described in Example 9.

FIG. 13 depicts results of a dynamic vapor sorption experiment conducted on Form D Crystalline Monosodium Salt of Compound I, as further described in Example 9.

FIG. 14 depicts an X-ray powder diffractogram of Form E Crystalline Disodium Salt of Compound I, as further described in Example 10.

FIG. 15 depicts a differential scanning calorimetry curve and a thermogravimetric analysis curve of Form E Crystalline Disodium Salt of Compound I, as further described in Example 10.

FIG. 16 depicts results of a dynamic vapor sorption experiment conducted on Form E Crystalline Disodium Salt of Compound I, as further described in Example 10.

FIG. 17 depicts an X-ray powder diffractogram of Form F Crystalline Disodium Salt of Compound I, as further described in Example 11.

FIG. 18 depicts a differential scanning calorimetry curve and a thermogravimetric analysis curve of Form F Crystalline Disodium Salt of Compound I, as further described in Example 11.

FIG. 19 depicts results of a dynamic vapor sorption experiment conducted on Form F Crystalline Disodium Salt of Compound I, as further described in Example 11.

FIG. 20 depicts an X-ray powder diffractogram of Form G Crystalline Monosodium Salt of Compound I, as further described in Example 12.

FIG. 21 depicts an X-ray powder diffractogram of Form H Crystalline Disodium Salt of Compound I, as further described in Example 13.

FIG. 22 depicts an X-ray powder diffractogram of Form I Crystalline Disodium Salt of Compound I, as further described in Example 14.

FIG. 23 depicts a differential scanning calorimetry curve and a thermogravimetric analysis curve of Form I Crystalline Disodium Salt of Compound I, as further described in Example 14.

FIG. 24 depicts results of a dynamic vapor sorption experiment conducted on Form I Crystalline Disodium Salt of Compound I, as further described in Example 14.

FIG. 25 depicts an X-ray powder diffractogram of Form J Crystalline Monosodium Salt of Compound I, as further described in Example 15.

FIG. 26 depicts an X-ray powder diffractogram of Form N Crystalline Monosodium Salt of Compound I, as further described in Example 16.

### DETAILED DESCRIPTION

The invention provides crystalline forms of compound I and salts of compound I.

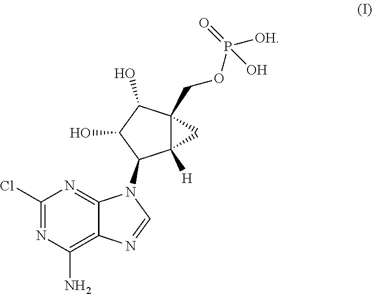 [SMILES: Nc1nc(Cl)nc2c1ncn2[C@H]1[C@H](O)[C@H](O)[C@]2(COP(=O)(O)O)C[C@H]12]

Also provided are pharmaceutical compositions, methods for making pharmaceutical compositions and methods for making the crystalline forms and salts of compound I, and use of the same in treating a disease or condition, such as heart failure. The crystalline forms may be characterized by physical characterization data, such as XPRD data. The practice of the present invention employs, unless otherwise indicated, conventional techniques of organic chemistry, pharmacology, molecular biology (including recombinant techniques), cell biology, biochemistry, and immunology. Such techniques are explained in the literature, such as in “Comprehensive Organic Synthesis” (B. M. Trost & I. Fleming, eds., 1991-1992); “Handbook of experimental immunology” (D. M. Weir & C. C. Blackwell, eds.); “Current protocols in molecular biology” (F. M. Ausubel et al., eds., 1987, and periodic updates); and “Current protocols in immunology” (J. E. Coligan et al., eds., 1991), each of which is herein incorporated by reference in its entirety.

Various aspects of the invention are set forth below in sections; however, aspects of the invention described in one particular section are not to be limited to any particular section. Further, when a variable is not accompanied by a definition, the previous definition of the variable controls.

### Definitions

Compounds of the present invention include those described generally herein, and are further illustrated by the classes, subclasses, and species disclosed herein. As used herein, the following definitions shall apply unless otherwise indicated. These definitions apply regardless of whether a term is used by itself or in combination with other terms, unless otherwise indicated. Hence, the definition of “alkyl” applies to “alkyl” as well as the “alkyl” portions of “—O-alkyl” etc. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “cycloaliphatic”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bicyclic ring” or “bicyclic ring system” refers to any bicyclic ring system, i.e. carbocyclic or heterocyclic, saturated or having one or more units of unsaturation, having one or more atoms in common between the two rings of the ring system. Thus, the term includes any permissible ring fusion, such as ortho-fused or spirocyclic. As used herein, the term “heterobicyclic” is a subset of “bicyclic” that requires that one or more heteroatoms are present in one or both rings of the bicycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms such as phosphates), boron, etc. In some embodiments, a bicyclic group has 7-12 ring members and 0-4 heteroatoms independently selected from nitrogen, oxygen, or sulfur. As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted.

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR●(as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “—(C₀ alkylene)-” refers to a bond. Accordingly, the term “—(C₀₋₃ alkylene)-” encompasses a bond (i.e., C₀) and a —(C₁₋₃ alkylene)- group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, quinolinyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where unless otherwise specified, the radical or point of attachment is on the heteroaromatic ring or on one of the rings to which the heteroaromatic ring is fused. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, and tetrahydroisoquinolinyl. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, 2-oxa-6-azaspiro[3.3]heptane, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Each optional substituent on a substitutable carbon is a monovalent substituent independently selected from halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄₀C(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —S(O)(NR^{∘})R^{∘}; —S(O)₂N═C(NR^{∘}₂)₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂.

Each R^{∘} is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted by a divalent substituent on a saturated carbon atom of R^{∘} selected from ═O and ═S; or each R^{∘} is optionally substituted with a monovalent substituent independently selected from halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●}.

Each R^{●} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{●} is unsubstituted or where preceded by halo is substituted only with one or more halogens; or wherein an optional substituent on a saturated carbon is a divalent substituent independently selected from ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, or a divalent substituent bound to vicinal substitutable carbons of an “optionally substituted” group is —O(CR*₂)₂₋₃O—, wherein each independent occurrence of R^{●} is selected from hydrogen, C₁₋₆ aliphatic or an unsubstituted 5-6-membered saturated, partially unsaturated, or aryl ring having 0-4 heteroatoms independently selected from nitrogen, oxygen, or sulfur.

When R* is C₁₋₆ aliphatic, R* is optionally substituted with halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{●} is unsubstituted or where preceded by halo is substituted only with one or more halogens.

An optional substituent on a substitutable nitrogen is independently —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic, unsubstituted —OPh, or an unsubstituted 5-6-membered saturated, partially unsaturated, or aryl ring having 0-4 heteroatoms independently selected from nitrogen, oxygen, or sulfur, or, two independent occurrences of Rt, taken together with their intervening atom(s) form an unsubstituted 3-12-membered saturated, partially unsaturated, or aryl mono- or bicyclic ring having 0-4 heteroatoms independently selected from nitrogen, oxygen, or sulfur; wherein when R^{†} is C₁₋₆ aliphatic, R^{†} is optionally substituted with halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{●} is unsubstituted or where preceded by halo is substituted only with one or more halogens.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are described in the literature. For example, S. M. Berge et al., describe pharmaceutically acceptable salts in detail in J. Pharmaceutical Sciences, 1977, 66, 1-19, incorporated herein by reference. Pharmaceutically acceptable salts of compounds can include those derived from suitable inorganic and organic acids and bases.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. The invention also include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

Diastereomeric mixtures can be separated into their individual diastereomers on the basis of their physical chemical differences by methods known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Alternatively, a particular enantiomer of a compound of the present invention may be prepared by asymmetric synthesis. Still further, where the molecule contains a basic functional group (such as amino) or an acidic functional group (such as carboxylic acid) diastereomeric salts are formed with an appropriate optically-active acid or base, followed by resolution of the diastereomers thus formed by fractional crystallization or chromatographic means known in the art, and subsequent recovery of the pure enantiomers.

Individual stereoisomers of the compounds of the invention may, for example, be substantially free of other isomers, or may be admixed, for example, as racemates or with all other, or other selected, stereoisomers. Chiral center(s) in a compound of the present invention can have the S or R configuration as defined by the IUPAC 1974 Recommendations. Further, to the extent a compound described herein may exist as a atropisomer (e.g., substituted biaryls), all forms of such atropisomer are considered part of this invention.

Chemical names, common names, and chemical structures may be used interchangeably to describe the same structure. If a chemical compound is referred to using both a chemical structure and a chemical name, and an ambiguity exists between the structure and the name, the structure predominates. It should also be noted that any carbon as well as heteroatom with unsatisfied valences in the text, schemes, examples and tables herein is assumed to have the sufficient number of hydrogen atom(s) to satisfy the valences.

The terms “a” and “an” as used herein mean “one or more” and include the plural unless the context is inappropriate.

Unless specified otherwise, the term “about” refers to within ±10% of the stated value. The invention encompasses embodiments where the value is within ±9%, ±8%, ±7%, ±6%, ±5%, ±4%, ±3%, ±2%, or ±1% of the stated value.

The term “alkyl” refers to a saturated straight or branched hydrocarbon, such as a straight or branched group of 1-12, 1-10, or 1-6 carbon atoms, referred to herein as C₁-C₁₂ alkyl, C₁-C₁₀ alkyl, and C₁-C₆ alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₃-C₆ cycloalkyl,” derived from a cycloalkane. Exemplary cycloalkyl groups include cyclohexyl, cyclopentyl, cyclobutyl, and cyclopropyl. The term “cycloalkylene” refers to a bivalent cycloalkyl group.

The term “haloalkyl” refers to an alkyl group that is substituted with at least one halogen. Exemplary haloalkyl groups include —CH₂F, —CHF₂, —CF₃, —CH₂CF₃, —CF₂CF₃, and the like. The term “haloalkylene” refers to a bivalent haloalkyl group.

The term “hydroxyalkyl” refers to an alkyl group that is substituted with at least one hydroxyl. Exemplary hydroxyalkyl groups include —CH₂CH₂OH, —C(H)(OH)CH₃, —CH₂C(H)(OH)CH₂CH₂OH, and the like.

The terms “alkenyl” and “alkynyl” are art-recognized and refer to unsaturated aliphatic groups analogous in length and possible substitution to the alkyls described above, but that contain at least one double or triple bond respectively.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. The term “haloalkoxyl” refers to an alkoxyl group that is substituted with at least one halogen. Exemplary haloalkoxyl groups include —OCH₂F, —OCHF₂, —OCF₃, —OCH₂CF₃, —OCF₂CF₃, and the like.

The term “oxo” is art-recognized and refers to a “═O” substituent. For example, a cyclopentane substituted with an oxo group is cyclopentanone.

The symbol “ [symbol] ” indicates a point of attachment.

When any substituent or variable occurs more than one time in any constituent or the compound of the invention, its definition on each occurrence is independent of its definition at every other occurrence, unless otherwise indicated.

One or more compounds of the invention may exist in unsolvated as well as solvated forms with pharmaceutically acceptable solvents such as water, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. “Hydrate” is a solvate wherein the solvent molecule is H₂O.

As used herein, the terms “subject” and “patient” are used interchangeably and refer to organisms to be treated by the methods of the present invention. Such organisms preferably include, but are not limited to, mammals (e.g., murines, simians, equines, bovines, porcines, canines, felines, and the like), and most preferably includes humans.

As used herein, the term “effective amount” refers to the amount of a compound sufficient to effect beneficial or desired results (e.g., a therapeutic, ameliorative, inhibitory or preventative result). An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, PA [1975].

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified.

### I. Crystalline Hydrate of Compound I

One aspect of the invention provides a crystalline hydrate of compound I:

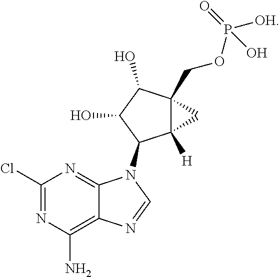 [SMILES: Nc1nc(Cl)nc2c1ncn2[C@H]1[C@H](O)[C@H](O)[C@]2(COP(=O)(O)O)C[C@H]12]

The compounds may be used in the pharmaceutical compositions and therapeutic methods described herein. Exemplary compounds are described in the following sections. Exemplary procedures for making the compounds are described in the Examples.

### A. Form A Crystalline Hydrate of Compound I

One aspect of the invention provides a compound that is Form A crystalline hydrate of Compound I. The Form A crystalline hydrate of Compound I may be more specifically defined according to XRPD data obtained on said form.

In certain embodiments, the crystalline form exhibits an X-ray powder diffraction pattern comprising peaks at the following diffraction angles (2θ): 10.3±0.2, 13.8±0.2, 19.0±0.2, 20.7±0.2, 21.8±0.2, 23.5±0.2, and 27.3±0.2. In certain embodiments, the X-ray powder diffraction pattern further comprises a peak at the following diffraction angle (2θ): 9.5±0.2. In certain embodiments, the X-ray powder diffraction pattern further comprises a peak at the following diffraction angle (2θ): 16.2±0.2. In certain embodiments, the X-ray powder diffraction pattern further comprises a peak at the following diffraction angle (2θ): 18.7±0.2. In certain embodiments, the X-ray powder diffraction pattern further comprises a peak at the following diffraction angle (2θ): 19.5±0.2. In certain embodiments, the X-ray powder diffraction pattern further comprises a peak at the following diffraction angle (2θ): 26.2±0.2. In certain embodiments, the X-ray powder diffraction pattern further comprises a peak at the following diffraction angle (2θ): 32.9±0.2.

In certain embodiments, the crystalline form exhibits an X-ray powder diffraction pattern comprising a peak at the following diffraction angle (2θ): 10.3±0.2. In certain embodiments, the crystalline form exhibits an X-ray powder diffraction pattern comprising a peak at the following diffraction angle (2θ): 13.8±0.2. In certain embodiments, the crystalline form exhibits an X-ray powder diffraction pattern comprising a peak at the following diffraction angle (2θ): 19.0±0.2. In certain embodiments, the crystalline form exhibits an X-ray powder diffraction pattern comprising a peak at the following diffraction angle (2θ): 20.7±0.2. In certain embodiments, the crystalline form exhibits an X-ray powder diffraction pattern comprising a peak at the following diffraction angle (2θ): 21.8±0.2. In certain embodiments, the crystalline form exhibits an X-ray powder diffraction pattern comprising a peak at the following diffraction angle (2θ): 23.5±0.2. In certain embodiments, the crystalline form exhibits an X-ray powder diffraction pattern comprising a peak at the following diffraction angle (2θ): 27.3±0.2.

In certain embodiments, the relative intensity of the peak at said diffraction angles (2θ) is at least 30%. In certain embodiments, the relative intensity of the peak at said diffraction angles (2θ) is at least 25%. In certain embodiments, the relative intensity of the peak at said diffraction angles (2θ) is at least 20%. In certain embodiments, the relative intensity of the peak at said diffraction angles (2θ) is at least 15%. In certain embodiments, the relative intensity of the peak at said diffraction angles (2θ) is at least 10%.

In certain embodiments, the crystalline form is characterized by the following X-ray powder diffraction pattern expressed in terms of diffraction angle 2θ, inter-planar distances d, and relative intensity (expressed as a percentage with respect to the most intense peak):

| Angle [2θ] | d-spacing [Å] | Relative Intensity [%] |
|---|---|---|
| 5.2 | 17.1 | 18 |
| 6.5 | 13.6 | 18 |
| 9.5 | 9.3 | 26 |
| 10.3 | 8.6 | 52 |
| 11.2 | 7.9 | 7 |
| 12.5 | 7.1 | 8 |
| 13.1 | 6.8 | 6 |
| 13.8 | 6.4 | 73 |
| 13.9 | 6.4 | 24 |
| 15.4 | 5.7 | 14 |
| 16.2 | 5.5 | 21 |
| 16.9 | 5.2 | 10 |
| 18.1 | 4.9 | 26 |
| 18.7 | 4.7 | 41 |
| 19.0 | 4.7 | 78 |
| 19.6 | 4.5 | 40 |
| 20.7 | 4.3 | 53 |
| 21.8 | 4.1 | 54 |
| 22.7 | 3.9 | 11 |
| 23.2 | 3.8 | 14 |
| 23.5 | 3.8 | 45 |
| 24.7 | 3.6 | 11 |
| 24.9 | 3.6 | 11 |
| 26.0 | 3.4 | 9 |
| 26.2 | 3.4 | 16 |
| 26.7 | 3.3 | 13 |
| 27.3 | 3.3 | 100 |
| 28.3 | 3.1 | 22 |
| 28.7 | 3.1 | 13 |
| 31.0 | 2.9 | 13 |
| 32.9 | 2.7 | 28 |
| 33.4 | 2.7 | 11 |
| 34.6 | 2.6 | 5 |
| 35.3 | 2.5 | 4 |
| 35.6 | 2.5 | 14 |
| 37.4 | 2.4 | 15 |
| 37.7 | 2.4 | 10 |
| 38.2 | 2.4 | 10 |
| 38.5 | 2.3 | 5 |
| 39.2 | 2.3 | 3 |

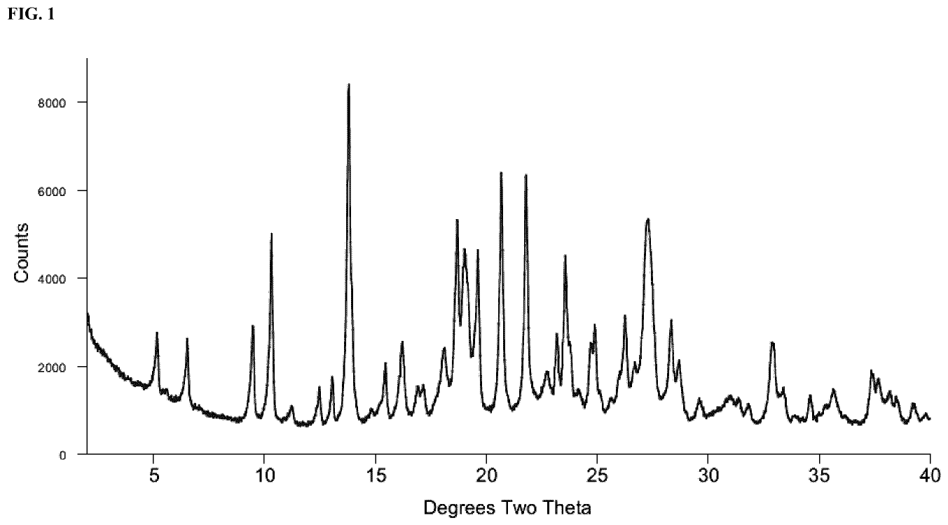

In certain embodiments, the crystalline form is characterized as having an X-ray powder diffraction pattern substantially as shown in FIG. 1.

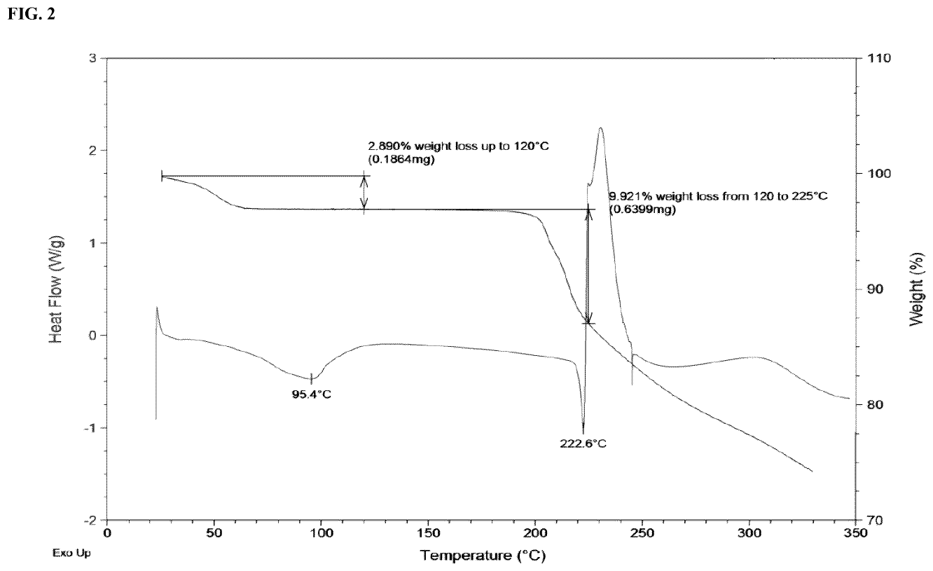

The Form A crystalline hydrate of Compound I may be more specifically defined according to melting point as determined by differential scanning calorimetry. Accordingly, in certain embodiments, the compound has a melting point as determined by differential scanning calorimetry in the range of from about 220 degrees Celsius to about 225 degrees Celsius. In certain embodiments, the compound has a melting point as determined by differential scanning calorimetry at about 223 degrees Celsius. In certain embodiments, the compound has a differential scanning calorimetry curve substantially the same as shown in FIG. 2.

The Form A crystalline hydrate of Compound I may be more specifically defined according to the mole ratio of water to compound I in the Form A crystalline hydrate of Compound I. In certain embodiments, the mole ratio of water to compound I is about 1:1. In certain embodiments, the mole ratio of water to compound I is 1:1.

### B. Form B Crystalline Hydrate of Compound I

Another aspect of the invention provides a compound that is Form B crystalline hydrate of Compound I. The Form B crystalline hydrate of Compound I may be more specifically defined according to XRPD data obtained on said form.

In certain embodiments, the crystalline form exhibits an X-ray powder diffraction pattern comprising peaks at the following diffraction angles (2θ): 8.9±0.2, 15.8±0.2, 21.3 0.2, 24.7±0.2, 26.4±0.2, 27.2±0.2, and 33.0±0.2. In certain embodiments, the X-ray powder diffraction pattern further comprises a peak at the following diffraction angle (2θ): 10.1±0.2. In certain embodiments, the X-ray powder diffraction pattern further comprises a peak at the following diffraction angle (2θ): 12.9±0.2. In certain embodiments, the X-ray powder diffraction pattern further comprises a peak at the following diffraction angle (2θ): 18.1±0.2. In certain embodiments, the X-ray powder diffraction pattern further comprises a peak at the following diffraction angle (2θ): 20.5±0.2. In certain embodiments, the X-ray powder diffraction pattern further comprises a peak at the following diffraction angle (2θ): 23.8±0.2. In certain embodiments, the X-ray powder diffraction pattern further comprises a peak at the following diffraction angle (2θ): 32.2±0.2.

In certain embodiments, the crystalline form exhibits an X-ray powder diffraction pattern comprising a peak at the following diffraction angle (2θ): 8.9±0.2. In certain embodiments, the crystalline form exhibits an X-ray powder diffraction pattern comprising a peak at the following diffraction angle (2θ): 15.8±0.2. In certain embodiments, the crystalline form exhibits an X-ray powder diffraction pattern comprising a peak at the following diffraction angle (2θ): 21.3±0.2. In certain embodiments, the crystalline form exhibits an X-ray powder diffraction pattern comprising a peak at the following diffraction angle (2θ): 24.7±0.2. In certain embodiments, the crystalline form exhibits an X-ray powder diffraction pattern comprising a peak at the following diffraction angle (2θ): 26.4±0.2. In certain embodiments, the crystalline form exhibits an X-ray powder diffraction pattern comprising a peak at the following diffraction angle (2θ): 27.2±0.2. In certain embodiments, the crystalline form exhibits an X-ray powder diffraction pattern comprising a peak at the following diffraction angle (2θ): 33.0±0.2.

In certain embodiments, the relative intensity of the peak at said diffraction angles (2θ) is at least 30%. In certain embodiments, the relative intensity of the peak at said diffraction angles (2θ) is at least 25%. In certain embodiments, the relative intensity of the peak at said diffraction angles (2θ) is at least 20%. In certain embodiments, the relative intensity of the peak at said diffraction angles (2θ) is at least 15%. In certain embodiments, the relative intensity of the peak at said diffraction angles (2θ) is at least 10%.

In certain embodiments, the crystalline form is characterized by the following X-ray powder diffraction pattern expressed in terms of diffraction angle 2θ, inter-planar distances d, and relative intensity (expressed as a percentage with respect to the most intense peak):

| Angle [2θ] | d-spacing [Å] | Relative Intensity [%] |
|---|---|---|
| 5.1 | 17.3 | 30 |
| 8.9 | 9.9 | 66 |
| 9.8 | 9.0 | 28 |
| 10.1 | 8.7 | 49 |
| 12.9 | 6.8 | 15 |
| 14.7 | 6.0 | 54 |
| 15.1 | 5.8 | 2 |
| 15.8 | 5.6 | 21 |
| 16.4 | 5.4 | 8 |
| 17.0 | 5.2 | 18 |
| 18.1 | 4.9 | 30 |
| 18.4 | 4.8 | 22 |
| 18.8 | 4.7 | 48 |
| 19.2 | 4.6 | 16 |
| 19.4 | 4.6 | 21 |
| 19.7 | 4.5 | 30 |
| 20.5 | 4.3 | 14 |
| 21.3 | 4.2 | 58 |
| 22.1 | 4.0 | 13 |
| 22.4 | 4.0 | 11 |
| 22.9 | 3.9 | 11 |
| 23.8 | 3.7 | 15 |
| 24.7 | 3.6 | 58 |
| 25.4 | 3.5 | 14 |
| 26.0 | 3.4 | 13 |
| 26.4 | 3.4 | 100 |
| 27.2 | 3.3 | 41 |
| 27.8 | 3.2 | 21 |
| 30.5 | 2.9 | 7 |
| 30.9 | 2.9 | 7 |
| 31.8 | 2.8 | 8 |
| 32.2 | 2.8 | 20 |
| 33.0 | 2.7 | 24 |
| 33.5 | 2.7 | 14 |
| 36.6 | 2.5 | 7 |
| 37.7 | 2.4 | 13 |
| 38.9 | 2.3 | 21 |

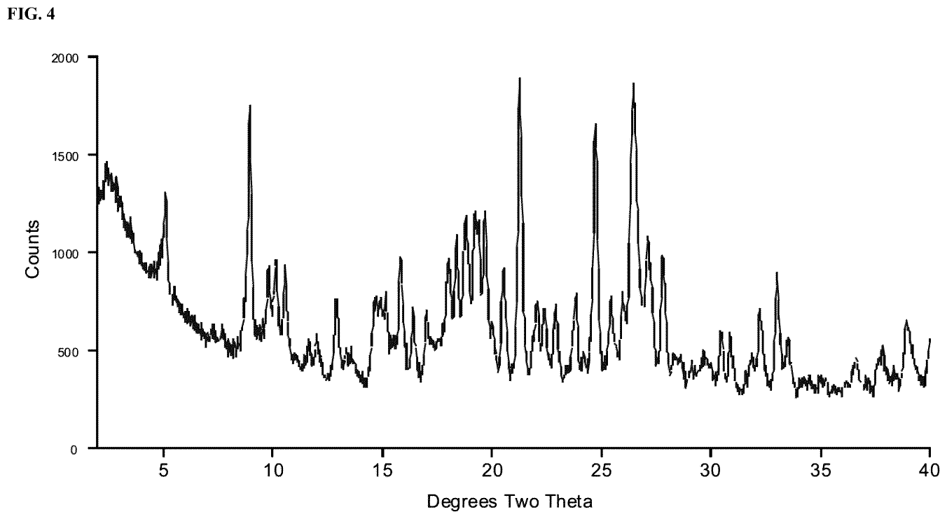

In certain embodiments, the crystalline form is characterized as having an X-ray powder diffraction pattern substantially as shown in FIG. 4.

The Form B crystalline hydrate of Compound I may be more specifically defined according to the mole ratio of water to compound I in the Form B crystalline hydrate of Compound I. In certain embodiments, the mole ratio of water to compound I is from 1.4:1 to 1.5:1. In certain embodiments, the mole ratio of water to compound I is about 1.5:1. In certain embodiments, the mole ratio of water to compound I is 1.5:1.

### II. Sodium Salt of Compound I

Another aspect of the invention provides a sodium salt of compound I:

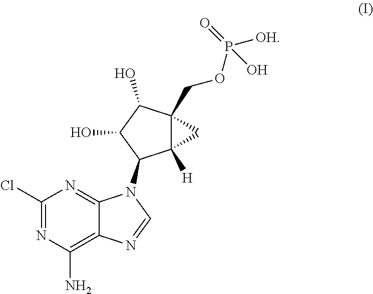 [SMILES: Nc1nc(Cl)nc2c1ncn2[C@H]1[C@H](O)[C@H](O)[C@]2(COP(=O)(O)O)C[C@H]12]

In certain embodiments, the compound is a monosodium salt of compound I. In certain embodiments, compound is a disodium salt of compound I.

In certain embodiments, the sodium salt of compound I is characterized according to stability when dissolved in an aqueous solution. For example, in certain embodiments, dissolution of the sodium salt of compound I in aqueous solution results in a solution where less than 1% (w/w) of the sodium salt of compound I degrades when stored for 24 hours at 50° C. In certain embodiments, dissolution of the sodium salt of compound I in aqueous solution results in a solution where less than 1% (w/w) of the sodium salt of compound I degrades when stored for 24 hours at 70° C.

In certain embodiments, the sodium salt compound is crystalline. Multiple different crystalline forms of a sodium salt of compound I are described herein below.

### A. Form A Crystalline Monosodium Salt of Compound I

One aspect of the invention provides Form A crystalline monosodium salt of compound I. The monosodium salt of compound I has the chemical formula:

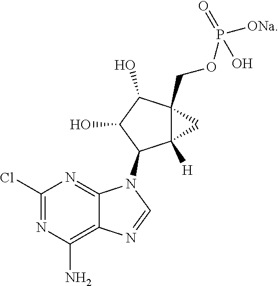

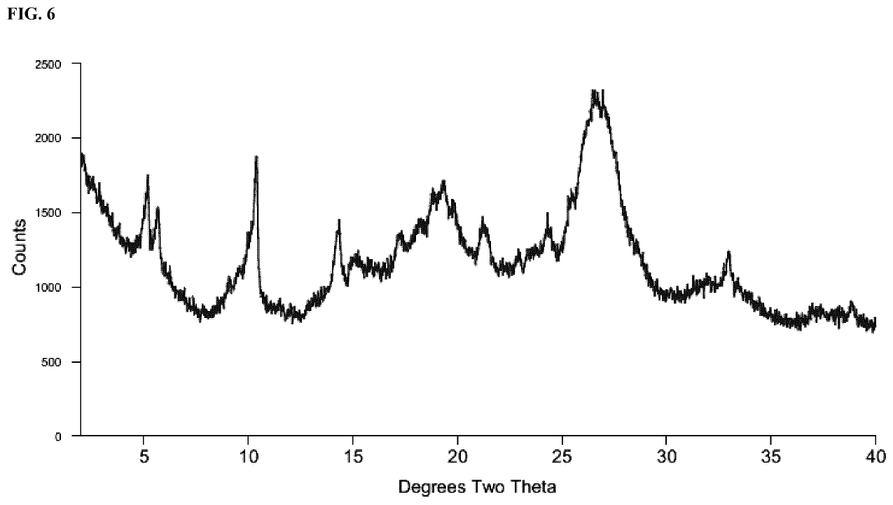

The Form A crystalline monosodium salt of compound I may be characterized according to the X-ray powder diffractogram in FIG. 6. Acccordingly, in certain embodiments, the Form A crystalline monosodium salt of compound I may be characterized as having an X-ray powder diffractogram substantially the same as the X-ray powder diffractogram provided in FIG. 6. In certain embodiments, the crystalline form exhibits an X-ray powder diffraction pattern comprising peaks at one or more of the following diffraction angles (2θ): 5.2±0.2, 5.7±0.2, 10.4±0.2, 14.3±0.2, 19.5±0.2, 21.2±0.2, 26.6±0.2, or 32.9±0.2. In certain embodiments, the crystalline form exhibits an X-ray powder diffraction pattern comprising peaks at at least five of the following diffraction angles (2θ): 5.2±0.2, 5.7±0.2, 10.4±0.2, 14.3±0.2, 19.5±0.2, 21.2±0.2, 26.6±0.2, or 32.9±0.2. In certain embodiments, the crystalline form exhibits an X-ray powder diffraction pattern comprising peaks at at least seven of the following diffraction angles (2θ): 5.2±0.2, 5.7±0.2, 10.4±0.2, 14.3±0.2, 19.5±0.2, 21.2±0.2, 26.6±0.2, or 32.9±0.2.

In certain embodiments, the crystalline form is characterized by the following X-ray powder diffraction pattern expressed in terms of diffraction angle 2θ, inter-planar distances d, and relative intensity (expressed as a percentage with respect to the most intense peak):

| Angle [2θ] | d-spacing [Å] | Relative Intensity [%] |
|---|---|---|
| 5.2 | 17.0 | 12 |
| 5.7 | 15.6 | 11 |
| 10.4 | 8.5 | 44 |
| 14.3 | 6.2 | 5 |
| 19.5 | 4.6 | 33 |
| 21.2 | 4.2 | 4 |
| 26.6 | 3.3 | 100 |
| 32.9 | 2.7 | 12 |

### B. Form B Crystalline Disodium Salt of Compound I

One aspect of the invention provides Form B crystalline disodium salt of compound I. The disodium salt of compound I has the chemical formula:

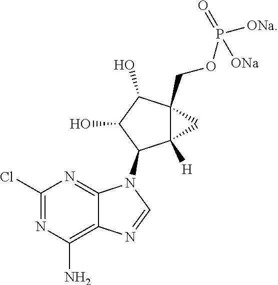

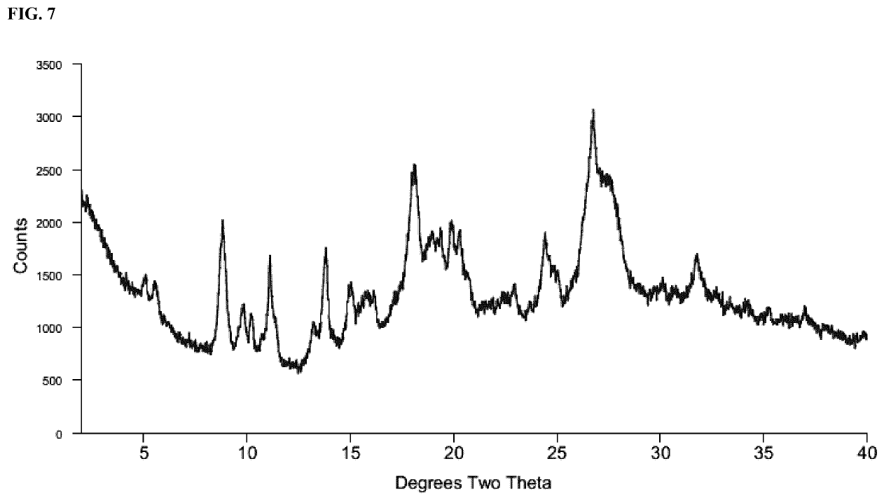

The Form B crystalline disodium salt of compound I may be characterized according to the X-ray powder diffractogram in FIG. 7. Acccordingly, in certain embodiments, the Form B crystalline disodium salt of compound I may be characterized as having an X-ray powder diffractogram substantially the same as the X-ray powder diffractogram provided in FIG. 7.

In certain embodiments, the crystalline form exhibits an X-ray powder diffraction pattern comprising peaks at one or more of the following diffraction angles (2θ): 5.4±0.2, 8.8 0.2, 9.8±0.2, 10.2±0.2, 11.1±0.2, 13.2±0.2, 13.8±0.2, 15.0±0.2, 18.1±0.2, 19.3±0.2, 19.9±0.2, 20.3±0.2, 24.4±0.2, 26.7±0.2, 27.6±0.2, or 31.8±0.2. In certain embodiments, the crystalline form exhibits an X-ray powder diffraction pattern comprising peaks at at least five of the following diffraction angles (2θ): 5.4±0.2, 8.8±0.2, 9.8±0.2, 10.2±0.2, 11.1±0.2, 13.2±0.2, 13.8±0.2, 15.0±0.2, 18.1±0.2, 19.3±0.2, 19.9±0.2, 20.3±0.2, 24.4±0.2, 26.7±0.2, 27.6±0.2, or 31.8±0.2. In certain embodiments, the crystalline form exhibits an X-ray powder diffraction pattern comprising peaks at at least seven of the following diffraction angles (2θ): 5.4±0.2, 8.8±0.2, 9.8±0.2, 10.2±0.2, 11.1±0.2, 13.2±0.2, 13.8±0.2, 15.0±0.2, 18.1±0.2, 19.3±0.2, 19.9±0.2, 20.3±0.2, 24.4±0.2, 26.7±0.2, 27.6±0.2, or 31.8±0.2.

In certain embodiments, the crystalline form is characterized by the following X-ray powder diffraction pattern expressed in terms of diffraction angle 2θ, along with inter-planar distances d):

| Angle [2θ] | d-spacing [Å] |
|---|---|
| 5.4 | 16.3 |
| 8.8 | 10.0 |
| 9.8 | 9.0 |
| 10.2 | 8.7 |
| 11.1 | 8.0 |
| 13.2 | 6.7 |
| 13.8 | 6.4 |
| 15.0 | 5.9 |
| 18.1 | 4.9 |
| 19.3 | 4.6 |
| 19.9 | 4.5 |
| 20.3 | 4.4 |
| 24.4 | 3.6 |
| 26.7 | 3.3 |
| 27.6 | 3.2 |
| 31.8 | 2.8 |

### C. Form C Crystalline Monosodium Salt of Compound I

One aspect of the invention provides Form C crystalline monosodium salt of compound I. The monosodium salt of compound I has the chemical formula:

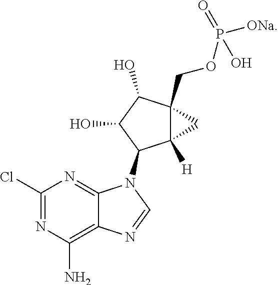

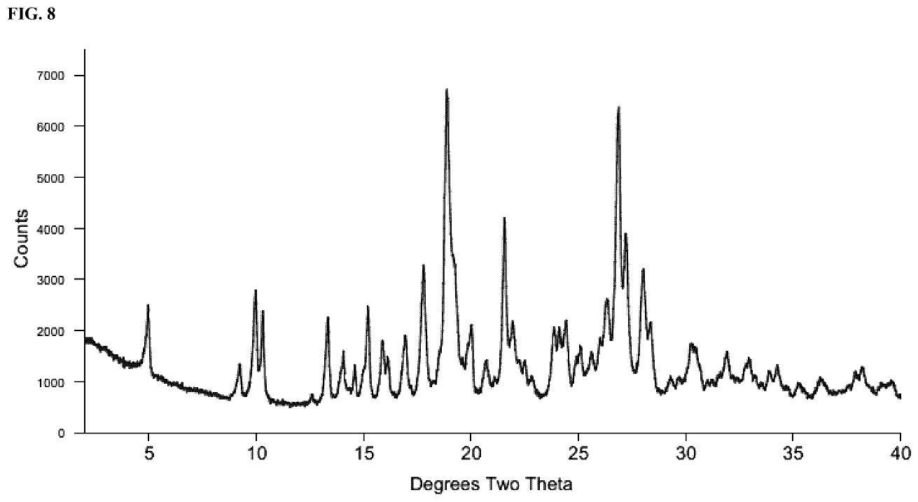

The Form C crystalline monosodium salt of compound I may be characterized according to the X-ray powder diffractogram in FIG. 8. Accordingly, in certain embodiments, the Form C crystalline monosodium salt of compound I may be characterized as having an X-ray powder diffractogram substantially the same as the X-ray powder diffractogram provided in FIG. 8. In certain embodiments, the crystalline form exhibits an X-ray powder diffraction pattern comprising peaks at one or more of the following diffraction angles (2θ): 5.0±0.2, 10.0±0.2, 10.3±0.2, 13.3±0.2, 14.0±0.2, 15.2±0.2, 15.9±0.2, 16.9±0.2, 17.8±0.2, 18.9±0.2, 19.2±0.2, 20.0±0.2, 21.6±0.2, 21.9±0.2, 23.8±0.2, 24.4±0.2, 26.0±0.2, 26.3±0.2, 26.9±0.2, 27.2±0.2, 28.0±0.2, 28.4±0.2, 30.4±0.2, 31.9±0.2, 32.9±0.2, or 34.3±0.2. In certain embodiments, the crystalline form exhibits an X-ray powder diffraction pattern comprising peaks at at least five of the following diffraction angles (2θ): 5.0±0.2, 10.0±0.2, 10.3±0.2, 13.3±0.2, 14.0±0.2, 15.2±0.2, 15.9±0.2, 16.9±0.2, 17.8±0.2, 18.9±0.2, 19.2±0.2, 20.0±0.2, 21.6±0.2, 21.9±0.2, 23.8±0.2, 24.4±0.2, 26.0±0.2, 26.3±0.2, 26.9±0.2, 27.2±0.2, 28.0±0.2, 28.4±0.2, 30.4±0.2, 31.9±0.2, 32.9±0.2, or 34.3±0.2. In certain embodiments, the crystalline form exhibits an X-ray powder diffraction pattern comprising peaks at at least seven of the following diffraction angles (2θ): 5.0±0.2, 10.0±0.2, 10.3±0.2, 13.3±0.2, 14.0±0.2, 15.2±0.2, 15.9±0.2, 16.9±0.2, 17.8±0.2, 18.9±0.2, 19.2±0.2, 20.0±0.2, 21.6±0.2, 21.9±0.2, 23.8±0.2, 24.4±0.2, 26.0±0.2, 26.3±0.2, 26.9±0.2, 27.2±0.2, 28.0±0.2, 28.4±0.2, 30.4±0.2, 31.9±0.2, 32.9±0.2, or 34.3±0.2.

In certain embodiments, the crystalline form is characterized by the following X-ray powder diffraction pattern expressed in terms of diffraction angle 2θ, inter-planar distances d, and relative intensity (expressed as a percentage with respect to the most intense peak):

| Angle [2θ] | d-spacing [Å] | Relative Intensity [%] |
|---|---|---|
| 5.0 | 17.8 | 23 |
| 9.2 | 9.6 | 11 |
| 10.0 | 8.9 | 39 |
| 10.3 | 8.6 | 24 |
| 13.3 | 6.6 | 23 |
| 14.0 | 6.3 | 15 |
| 14.6 | 6.1 | 5 |
| 15.2 | 5.8 | 25 |
| 15.9 | 5.6 | 12 |
| 16.1 | 5.5 | 7 |
| 16.9 | 5.2 | 15 |
| 17.8 | 5.0 | 40 |
| 18.5 | 4.8 | 10 |
| 18.9 | 4.7 | 100 |
| 19.2 | 4.6 | 52 |
| 20.0 | 4.4 | 26 |
| 20.7 | 4.3 | 6 |
| 21.6 | 4.1 | 38 |
| 21.9 | 4.1 | 27 |
| 22.5 | 3.9 | 8 |
| 23.8 | 3.7 | 14 |
| 24.1 | 3.7 | 10 |
| 24.4 | 3.6 | 15 |
| 24.9 | 3.6 | 4 |
| 25.1 | 3.5 | 7 |
| 25.6 | 3.5 | 7 |
| 26.0 | 3.4 | 8 |
| 26.3 | 3.4 | 37 |
| 26.9 | 3.3 | 94 |
| 27.2 | 3.3 | 45 |
| 28.0 | 3.2 | 35 |
| 28.4 | 3.1 | 16 |
| 30.4 | 2.9 | 22 |
| 31.9 | 2.8 | 16 |
| 32.9 | 2.7 | 19 |
| 34.3 | 2.6 | 13 |
| 36.3 | 2.5 | 4 |
| 37.9 | 2.4 | 5 |
| 38.2 | 2.4 | 10 |

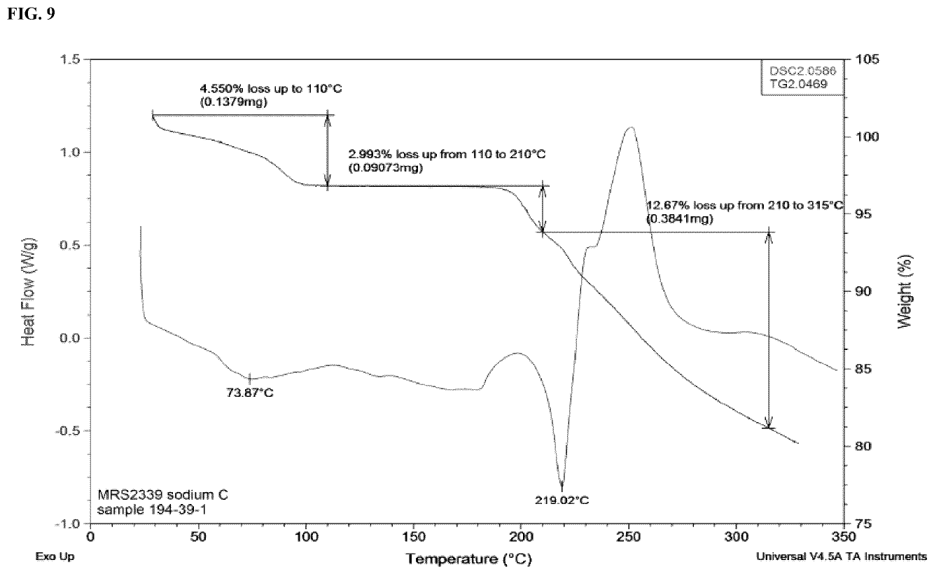

In certain embodiments, the Form C crystalline monosodium salt of compound I may be characterized according to differential scanning calorimetry and/or a thermogravimetric analysis. In certain embodiments, the Form C crystalline monosodium salt of compound I may be characterized as having a differential scanning calorimetry curve and/or a thermogravimetric analysis curve substantially the same as that provided in FIG. 9.

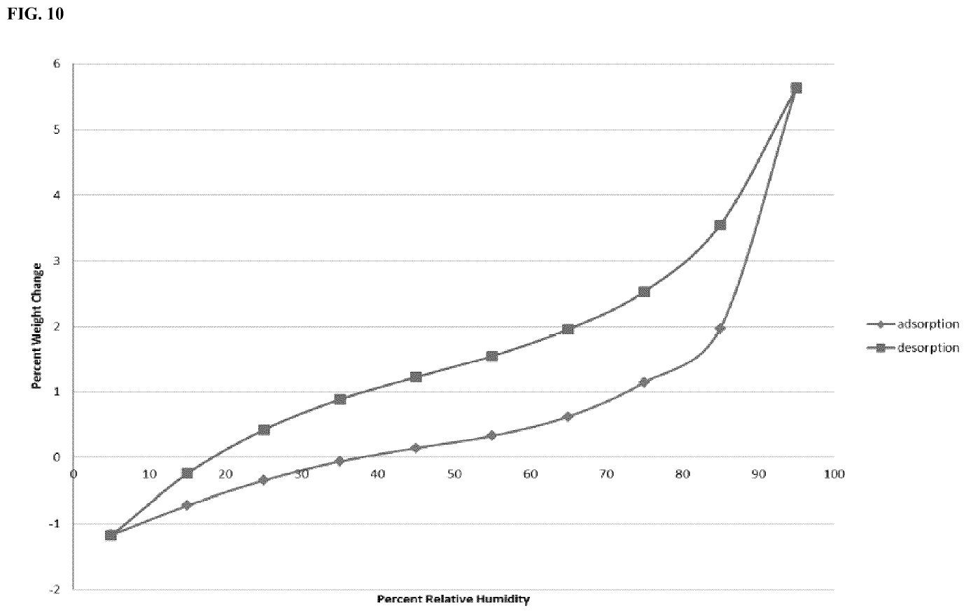

In certain embodiments, the Form C crystalline monosodium salt of compound I may be characterized according to dynamic vapor sorption. In certain embodiments, the Form C crystalline monosodium salt of compound I may be characterized according to dynamic vapor sorption curve that is substantially the same as that provided in FIG. 10.

### D. Form D Crystalline Monosodium Salt of Compound I

One aspect of the invention provides Form D crystalline monosodium salt of compound I. The monosodium salt of compound I has the chemical formula:

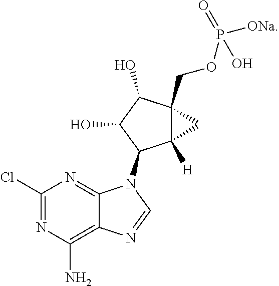

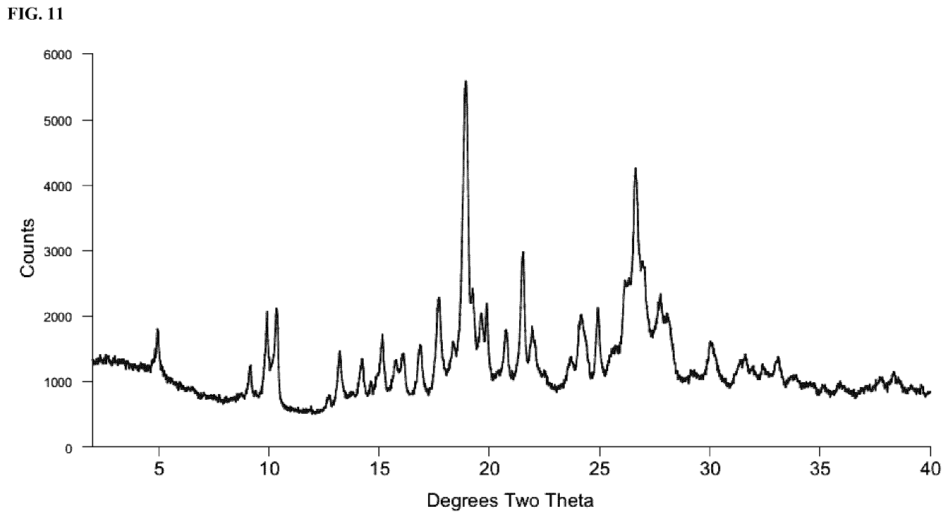

The Form D crystalline monosodium salt of compound I may be characterized according to the X-ray powder diffractogram provided in FIG. 11. Acccordingly, in certain embodiments, the Form D crystalline monosodium salt of compound I may be characterized as having an X-ray powder diffractogram substantially the same as the X-ray powder diffractogram provided in FIG. 11. In certain embodiments, the crystalline form exhibits an X-ray powder diffraction pattern comprising peaks at one or more of the following diffraction angles (2θ): 5.0±0.2, 9.2±0.2, 9.9±0.2, 10.4±0.2, 13.2±0.2, 15.1±0.2, 17.7±0.2, 18.4±0.2, 18.9±0.2, 19.2±0.2, 19.6±0.2, 21.5±0.2, 21.9±0.2, 24.1±0.2, 26.6±0.2, 26.8±0.2, or 28.1±0.2. In certain embodiments, the crystalline form exhibits an X-ray powder diffraction pattern comprising peaks at at least five of the following diffraction angles (2θ): 5.0±0.2, 9.2±0.2, 9.9±±0.2, 10.4±0.2, 13.2±0.2, 15.1±0.2, 17.7±0.2, 18.4±0.2, 18.9±0.2, 19.2±0.2, 19.6±0.2, 21.5±0.2, 21.9±0.2, 24.1±0.2, 26.6±0.2, 26.8±0.2, or 28.1±0.2. In certain embodiments, the crystalline form exhibits an X-ray powder diffraction pattern comprising peaks at at least seven of the following diffraction angles (2θ): 5.0±0.2, 9.2±0.2, 9.9±0.2, 10.4±0.2, 13.2±0.2, 15.1±0.2, 17.7±0.2, 18.4±0.2, 18.9±0.2, 19.2±0.2, 19.6±0.2, 21.5±0.2, 21.9±0.2, 24.1±0.2, 26.6±0.2, 26.8±0.2, or 28.1±0.2.

In certain embodiments, the crystalline form is characterized by the following X-ray powder diffraction pattern expressed in terms of diffraction angle 2θ, inter-planar distances d, and relative intensity (expressed as a percentage with respect to the most intense peak):

| Angle [2θ] | d-spacing [Å] | Relative Intensity [%] |
|---|---|---|
| 5.0 | 17.8 | 14 |
| 9.2 | 9.6 | 14 |
| 9.9 | 8.9 | 18 |
| 10.4 | 8.5 | 35 |
| 13.2 | 6.7 | 14 |
| 14.2 | 6.2 | 10 |
| 15.1 | 5.8 | 17 |
| 15.7 | 5.6 | 5 |
| 16.1 | 5.5 | 9 |
| 16.8 | 5.3 | 7 |
| 17.7 | 5.0 | 20 |
| 18.4 | 4.8 | 17 |
| 18.9 | 4.7 | 80 |
| 19.2 | 4.6 | 13 |
| 19.6 | 4.5 | 15 |
| 19.9 | 4.5 | 8 |
| 20.8 | 4.3 | 9 |
| 21.5 | 4.1 | 24 |
| 21.9 | 4.0 | 13 |
| 23.6 | 3.8 | 4 |
| 24.1 | 3.7 | 20 |
| 24.9 | 3.6 | 10 |
| 26.1 | 3.4 | 8 |
| 26.6 | 3.3 | 19 |
| 26.8 | 3.3 | 100 |
| 27.8 | 3.2 | 8 |
| 28.1 | 3.2 | 38 |
| 30.0 | 3.0 | 10 |
| 31.4 | 2.8 | 13 |
| 32.4 | 2.8 | 5 |
| 33.1 | 2.7 | 7 |
| 33.8 | 2.6 | 4 |
| 38.4 | 2.3 | 8 |

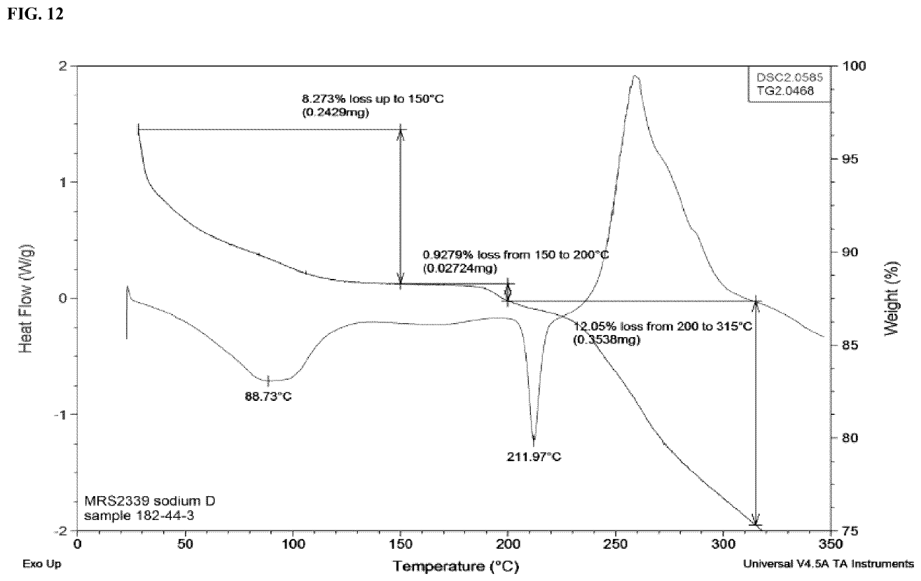

In certain embodiments, the Form D crystalline monosodium salt of compound I may be characterized according to differential scanning calorimetry and/or a thermogravimetric analysis. In certain embodiments, the Form D crystalline monosodium salt of compound I may be characterized as having a differential scanning calorimetry curve and/or a thermogravimetric analysis curve that is substantially the same as that provided in FIG. 12.

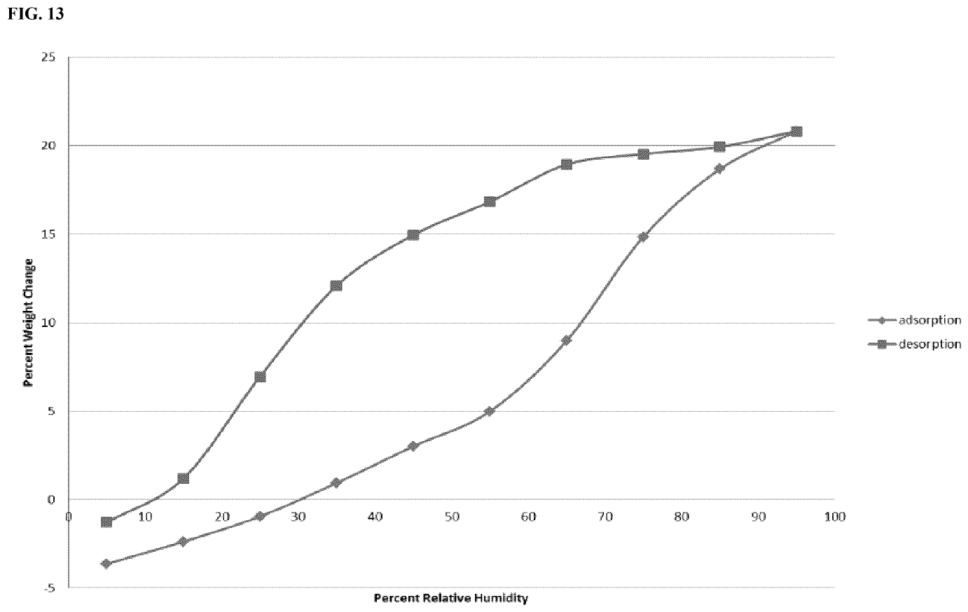

In certain embodiments, the Form D crystalline monosodium salt of compound I may be characterized according to dynamic vapor sorption. In certain embodiments, the Form D crystalline monosodium salt of compound I may be characterized as having a dynamic vapor sorption curve substantially the same as that provided in FIG. 13.

### E. Form E Crystalline Disodium Salt of Compound I

One aspect of the invention provides Form E crystalline disodium salt of compound I. The disodium salt of compound I has the chemical formula:

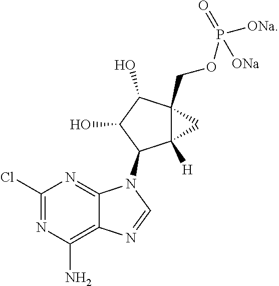

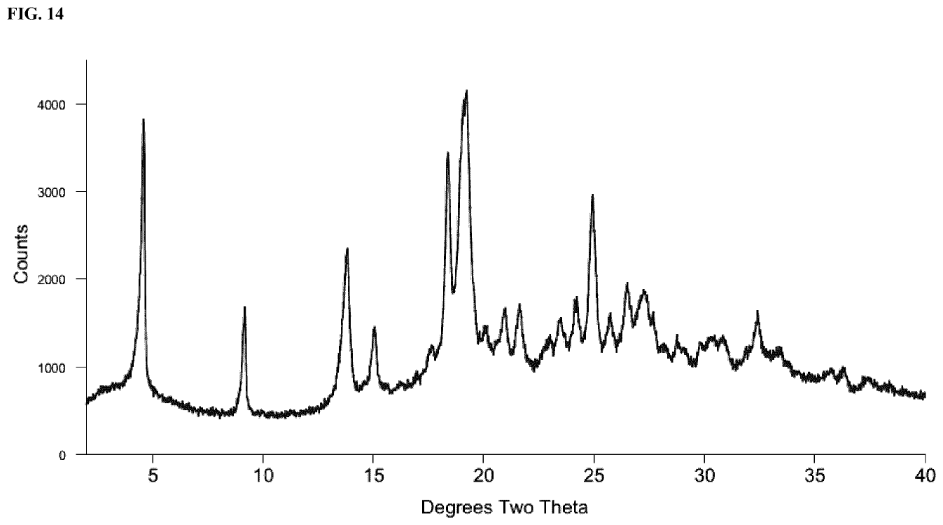

The Form E crystalline disodium salt of compound I may be characterized according to the X-ray powder diffractogram provided in FIG. 14. Accordingly, in certain embodiments, the Form E crystalline disodium salt of compound I may be characterized as having an X-ray powder diffractogram substantially the same as the X-ray powder diffractogram provided in FIG. 14. In certain embodiments, the crystalline form exhibits an X-ray powder diffraction pattern comprising peaks at one or more of the following diffraction angles (2θ): 4.6±0.2, 9.2±0.2, 13.8±0.2, 15.0±0.2, 17.6±0.2, 18.4±0.2, 19.2±0.2, 24.9±0.2, 26.5±0.2, or 27.2±0.2. In certain embodiments, the crystalline form exhibits an X-ray powder diffraction pattern comprising peaks at at least five of the following diffraction angles (2θ): 4.6±0.2, 9.2±0.2, 13.8±0.2, 15.0±0.2, 17.6±0.2, 18.4±0.2, 19.2±0.2, 24.9±0.2, 26.5±0.2, or 27.2±0.2. In certain embodiments, the crystalline form exhibits an X-ray powder diffraction pattern comprising peaks at at least seven of the following diffraction angles (2θ): 4.6±0.2, 9.2±0.2, 13.8±0.2, 15.0±0.2, 17.6±0.2, 18.4±0.2, 19.2±0.2, 24.9±0.2, 26.5±0.2, or 27.2±0.2.

In certain embodiments, the crystalline form is characterized by the following X-ray powder diffraction pattern expressed in terms of diffraction angle 2θ, inter-planar distances d, and relative intensity (expressed as a percentage with respect to the most intense peak):

| Angle [2θ] | d-spacing [Å] | Relative Intensity [%] |
|---|---|---|
| 4.6 | 19.2 | 50 |
| 9.2 | 9.6 | 17 |
| 13.8 | 6.4 | 42 |
| 15.0 | 5.9 | 14 |
| 17.6 | 5.0 | 12 |
| 18.4 | 4.8 | 34 |
| 19.2 | 4.6 | 100 |
| 20.1 | 4.4 | 3 |
| 20.9 | 4.2 | 7 |
| 21.6 | 4.1 | 7 |
| 23.4 | 3.8 | 4 |
| 24.2 | 3.7 | 7 |
| 24.9 | 3.6 | 28 |
| 25.7 | 3.5 | 4 |
| 26.5 | 3.4 | 11 |
| 27.2 | 3.3 | 24 |
| 30.9 | 2.9 | 8 |
| 32.4 | 2.8 | 9 |
| 33.4 | 2.7 | 8 |

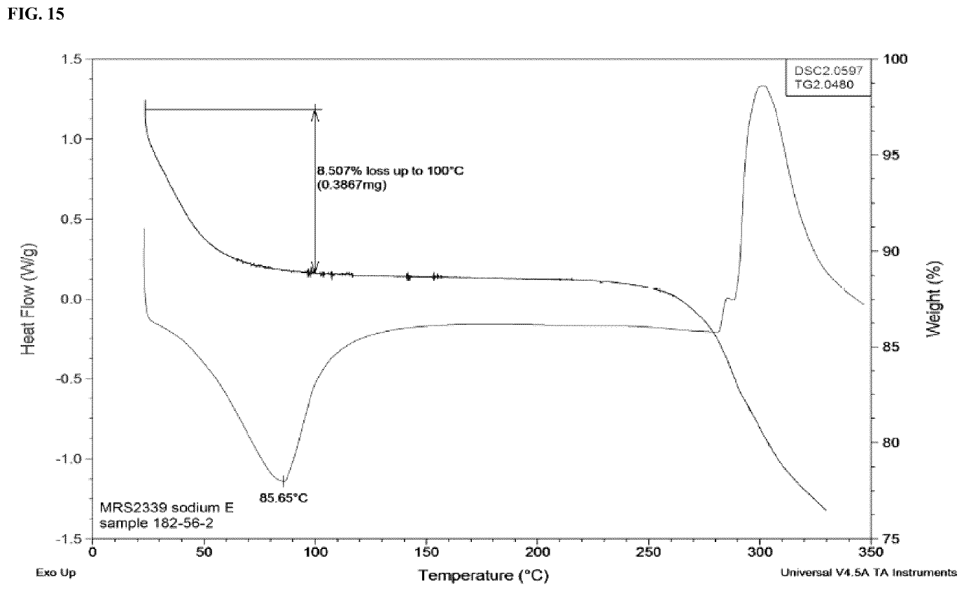

In certain embodiments, the Form E crystalline disodium salt of compound I may be characterized according to differential scanning calorimetry and/or a thermogravimetric analysis. In certain embodiments, the Form E crystalline disodium salt of compound I may be characterized as having a differential scanning calorimetry curve and/or a thermogravimetric analysis curve that is substantially the same as that provided in FIG. 15.

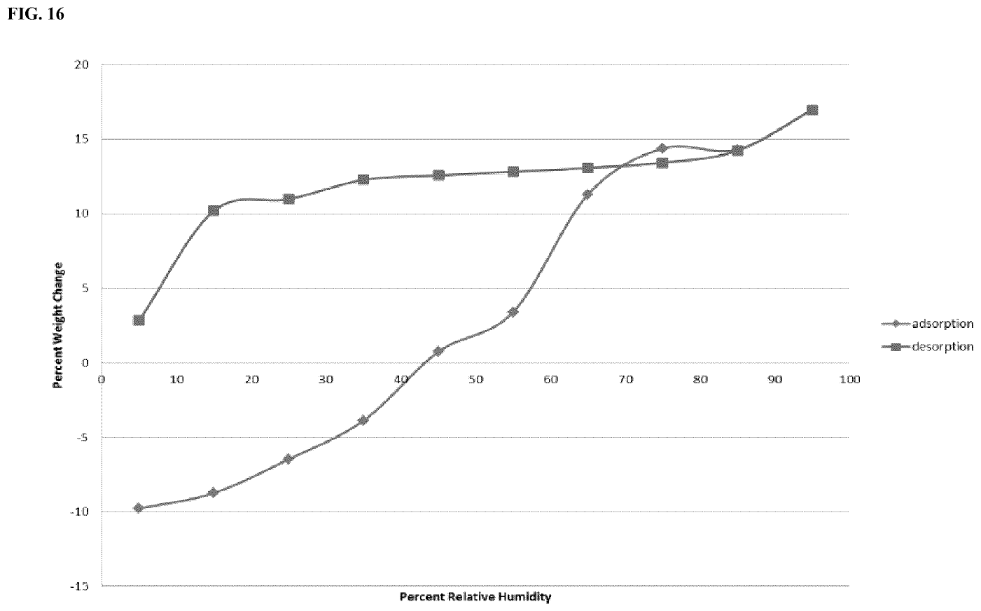

In certain embodiments, the Form E crystalline disodium salt of compound I may be characterized according to dynamic vapor sorption. In certain embodiments, the Form E crystalline disodium salt of compound I may be characterized as having a dynamic vapor sorption curve substantially the same as that provided in FIG. 16.

### F. Form F Crystalline Disodium Salt of Compound I

One aspect of the invention provides Form F crystalline disodium salt of compound I. The disodium salt of compound I has the chemical formula:

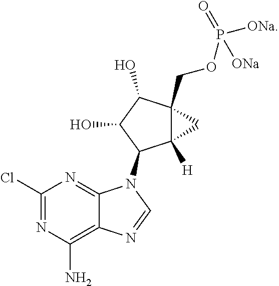

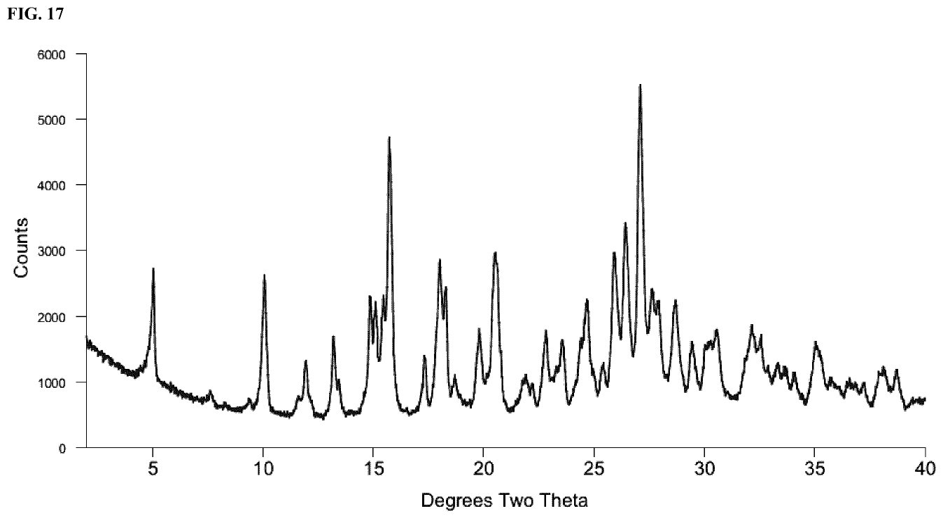

The Form F crystalline disodium salt of compound I may be characterized according to the X-ray powder diffractogram provided in FIG. 17. Acccordingly, in certain embodiments, the Form F crystalline disodium salt of compound I may be characterized as having an X-ray powder diffractogram substantially the same as the X-ray powder diffractogram provided in FIG. 17. In certain embodiments, the crystalline form exhibits an X-ray powder diffraction pattern comprising peaks at one or more of the following diffraction angles (2θ): 5.0±0.2, 10.1±0.2, 12.0±0.2, 13.2±0.2, 14.8±0.2, 15.1±0.2, 15.4±0.2, 15.7±0.2, 18.0±0.2, 18.3±0.2, 19.7±0.2, 20.5±0.2, 21.9±0.2, 22.8±0.2, 23.5±0.2, 24.4±0.2, 24.7±0.2, 25.4±0.2, 25.9±0.2, 26.4±0.2, 27.1±0.2, 27.6±0.2, 27.9±0.2, 28.7±0.2, 29.4±0.2, 30.5±0.2, 32.1±0.2, or 32.5±0.2. In certain embodiments, the crystalline form exhibits an X-ray powder diffraction pattern comprising peaks at at least five of the following diffraction angles (2θ): 5.0±0.2, 10.1±0.2, 12.0±0.2, 13.2±0.2, 14.8±0.2, 15.1±0.2, 15.4±0.2, 15.7±0.2, 18.0±0.2, 18.3±0.2, 19.7±0.2, 20.5±0.2, 21.9±0.2, 22.8±0.2, 23.5±0.2, 24.4±0.2, 24.7±0.2, 25.4±0.2, 25.9±0.2, 26.4±0.2, 27.1±0.2, 27.6±0.2, 27.9±0.2, 28.7±0.2, 29.4±0.2, 30.5±0.2, 32.1±0.2, or 32.5±0.2. In certain embodiments, the crystalline form exhibits an X-ray powder diffraction pattern comprising peaks at at least seven of the following diffraction angles (2θ): 5.0±0.2, 10.1±0.2, 12.0±0.2, 13.2±0.2, 14.8±0.2, 15.1±0.2, 15.4±0.2, 15.7±0.2, 18.0±0.2, 18.3±0.2, 19.7±0.2, 20.5±0.2, 21.9±0.2, 22.8±0.2, 23.5±0.2, 24.4±0.2, 24.7±0.2, 25.4±0.2, 25.9±0.2, 26.4±0.2, 27.1±0.2, 27.6±0.2, 27.9±0.2, 28.7±0.2, 29.4±0.2, 30.5±0.2, 32.1±0.2, or 32.5±0.2.

In certain embodiments, the crystalline form is characterized by the following X-ray powder diffraction pattern expressed in terms of diffraction angle 2θ, inter-planar distances d, and relative intensity (expressed as a percentage with respect to the most intense peak):

| Angle [2θ] | d-spacing [Å] | Relative Intensity [%] |
|---|---|---|
| 5.0 | 17.5 | 38 |
| 7.6 | 11.6 | 3 |
| 10.1 | 8.8 | 48 |
| 11.7 | 7.6 | 8 |
| 12.0 | 7.4 | 17 |
| 13.2 | 6.7 | 29 |
| 13.4 | 6.6 | 4 |
| 14.8 | 6.0 | 24 |
| 15.1 | 5.9 | 30 |
| 15.4 | 5.7 | 26 |
| 15.7 | 5.6 | 100 |
| 17.3 | 5.1 | 11 |
| 18.0 | 4.9 | 48 |
| 18.3 | 4.9 | 25 |
| 18.7 | 4.7 | 8 |
| 19.7 | 4.5 | 24 |
| 20.5 | 4.3 | 66 |
| 21.9 | 4.1 | 13 |
| 22.8 | 3.9 | 21 |
| 23.5 | 3.8 | 23 |
| 24.4 | 3.7 | 10 |
| 24.7 | 3.6 | 27 |
| 25.4 | 3.5 | 3 |
| 25.9 | 3.4 | 45 |
| 26.4 | 3.4 | 52 |
| 27.1 | 3.3 | 91 |
| 27.6 | 3.2 | 25 |
| 27.9 | 3.2 | 32 |
| 28.7 | 3.1 | 30 |
| 29.4 | 3.0 | 11 |
| 30.5 | 2.9 | 45 |
| 32.1 | 2.8 | 34 |
| 32.5 | 2.8 | 18 |
| 32.9 | 2.7 | 9 |
| 33.3 | 2.7 | 12 |
| 33.6 | 2.7 | 7 |
| 34.1 | 2.6 | 5 |
| 35.1 | 2.6 | 25 |
| 35.8 | 2.5 | 3 |
| 36.5 | 2.5 | 3 |
| 36.8 | 2.4 | 1 |
| 37.2 | 2.4 | 2 |
| 38.0 | 2.4 | 20 |
| 38.7 | 2.3 | 10 |

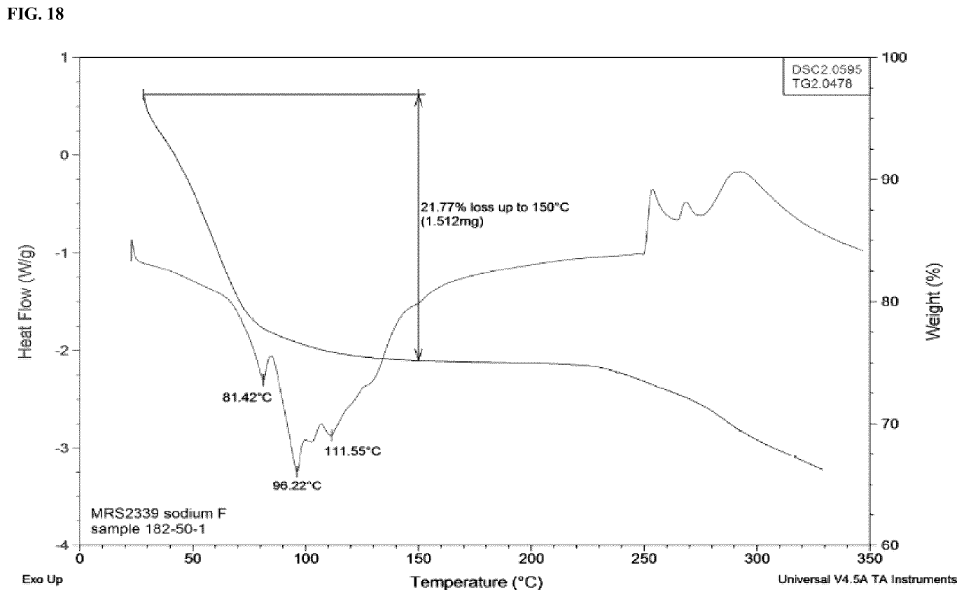

In certain embodiments, the Form F crystalline disodium salt of compound I may be characterized according to differential scanning calorimetry and/or a thermogravimetric analysis. In certain embodiments, the Form F crystalline disodium salt of compound I may be characterized as having a differential scanning calorimetry curve and/or a thermogravimetric analysis curve that is substantially the same as that provided in FIG. 18.

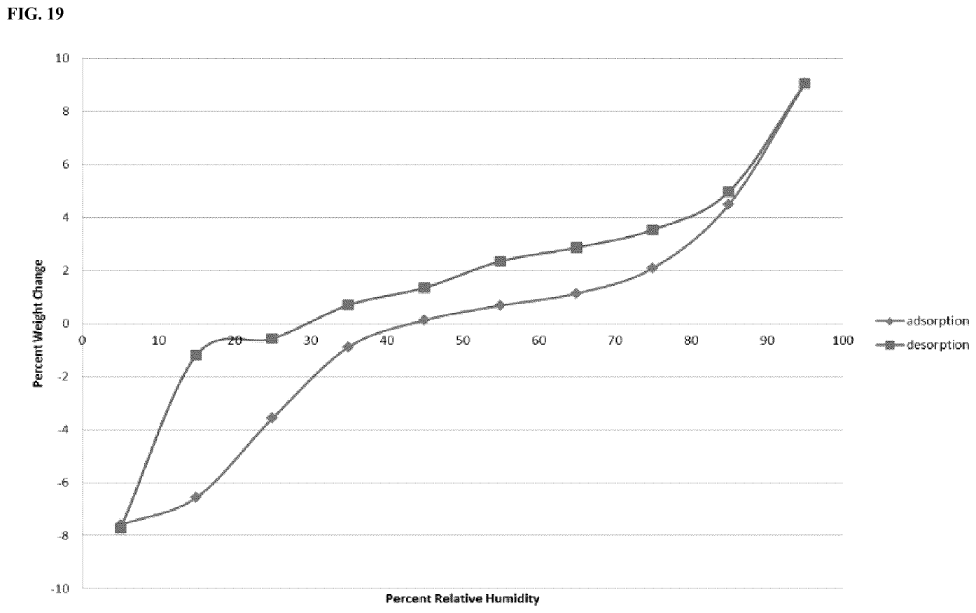

In certain embodiments, the Form F crystalline disodium salt of compound I may be characterized according to dynamic vapor sorption. In certain embodiments, the the Form F crystalline disodium salt of compound I may be characterized as having a dynamic vapor sorption curve substantially the same as that provided in FIG. 19.

### G. Form G Crystalline Monosodium Salt of Compound I

One aspect of the invention provides Form G crystalline monosodium salt of compound I. The monosodium salt of compound I has the chemical formula:

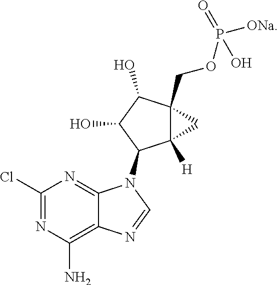

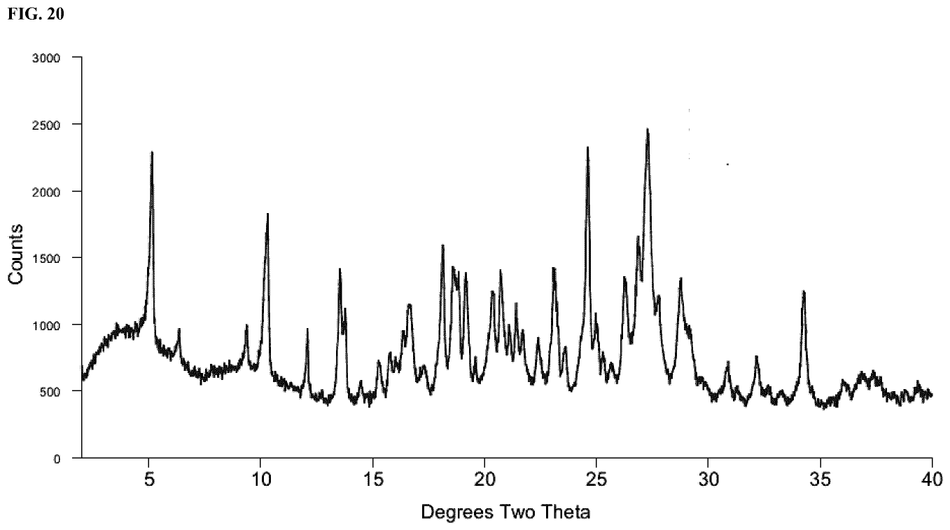

The Form G crystalline monosodium salt of compound I may be characterized according to the X-ray powder diffractogram provided in FIG. 20. Acccordingly, in certain embodiments, the Form G crystalline monosodium salt of compound I may be characterized as having an X-ray powder diffractogram substantially the same as the X-ray powder diffractogram provided in FIG. 20. In certain embodiments, the crystalline form exhibits an X-ray powder diffraction pattern comprising peaks at one or more of the following diffraction angles (2θ): 5.2±0.2, 9.4±0.2, 10.3±0.2, 12.1±0.2, 13.5±0.2, 13.8±0.2, 16.7±0.2, 18.1±0.2, 18.6±0.2, 18.8±0.2, 19.2±0.2, 20.4±0.2, 20.7±0.2, 23.1±0.2, 24.6±0.2, 25.0±0.2, 26.2±0.2, 26.9±0.2, 27.3±0.2, 27.8±0.2, 28.7±0.2, 29.2±0.2, 30.8±0.2, 32.2±0.2, or 34.2±0.2. In certain embodiments, the crystalline form exhibits an X-ray powder diffraction pattern comprising peaks at at least five of the following diffraction angles (2θ): 5.2±0.2, 9.4±0.2, 10.3±0.2, 12.1±0.2, 13.5±0.2, 13.8±0.2, 16.7±0.2, 18.1±0.2, 18.6±0.2, 18.8±0.2, 19.2±0.2, 20.4±0.2, 20.7±0.2, 23.1±0.2, 24.6±0.2, 25.0±0.2, 26.2±0.2, 26.9±0.2, 27.3±0.2, 27.8±0.2, 28.7±0.2, 29.2±0.2, 30.8±0.2, 32.2±0.2, or 34.2±0.2. In certain embodiments, the crystalline form exhibits an X-ray powder diffraction pattern comprising peaks at at least seven of the following diffraction angles (2θ): 5.2±0.2, 9.4±0.2, 10.3±0.2, 12.1±0.2, 13.5±0.2, 13.8±0.2, 16.7±0.2, 18.1±0.2, 18.6±0.2, 18.8±0.2, 19.2±0.2, 20.4±0.2, 20.7±0.2, 23.1±0.2, 24.6±0.2, 25.0±0.2, 26.2±0.2, 26.9±0.2, 27.3±0.2, 27.8±0.2, 28.7±0.2, 29.2±0.2, 30.8±0.2, 32.2±0.2, or 34.2±0.2.

In certain embodiments, the crystalline form is characterized by the following X-ray powder diffraction pattern expressed in terms of diffraction angle 2θ, inter-planar distances d, and relative intensity (expressed as a percentage with respect to the most intense peak):

| Angle [2θ] | d-spacing [Å] | Relative Intensity [%] |
|---|---|---|
| 5.2 | 17.1 | 58 |
| 9.4 | 9.4 | 26 |
| 10.3 | 8.6 | 69 |
| 12.1 | 7.3 | 11 |
| 13.5 | 6.5 | 25 |
| 13.8 | 6.4 | 21 |
| 16.7 | 5.3 | 82 |
| 18.1 | 4.9 | 29 |
| 18.6 | 4.8 | 25 |
| 18.8 | 4.7 | 19 |
| 19.2 | 4.6 | 20 |
| 20.4 | 4.4 | 20 |
| 20.7 | 4.3 | 23 |
| 21.1 | 4.2 | 7 |
| 21.4 | 4.1 | 10 |
| 21.7 | 4.1 | 7 |
| 22.4 | 4.0 | 5 |
| 23.1 | 3.8 | 31 |
| 23.6 | 3.8 | 5 |
| 24.6 | 3.6 | 72 |
| 25.0 | 3.6 | 17 |
| 26.2 | 3.4 | 34 |
| 26.9 | 3.3 | 54 |
| 27.3 | 3.3 | 100 |
| 27.8 | 3.2 | 20 |
| 28.7 | 3.1 | 29 |
| 29.2 | 3.1 | 25 |
| 30.8 | 2.9 | 9 |
| 32.2 | 2.8 | 19 |
| 34.2 | 2.6 | 39 |
| 37.0 | 2.4 | 22 |

### H. Form H Crystalline Disodium Salt of Compound I

One aspect of the invention provides Form H crystalline disodium salt of compound I. The disodium salt of compound I has the chemical formula:

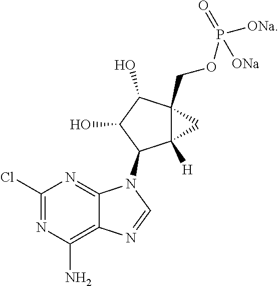

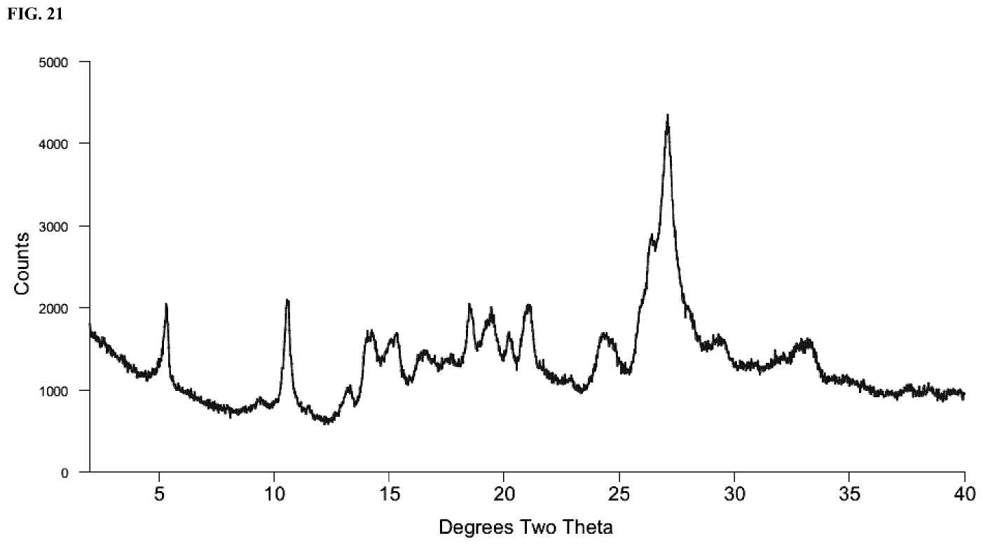

The Form H crystalline disodium salt of compound I may be characterized according to the X-ray powder diffractogram provided in FIG. 21. Accordingly, in certain embodiments, the Form H crystalline disodium salt of compound I may be characterized as having an X-ray powder diffractogram substantially the same as the X-ray powder diffractogram provided in FIG. 21. In certain embodiments, the crystalline form exhibits an X-ray powder diffraction pattern comprising peaks at one or more of the following diffraction angles (2θ): 5.3±0.2, 10.6±0.2, 14.1±0.2, 15.3±0.2, 18.5±0.2, 19.4±0.2, 21.0±0.2, 24.4±0.2, 26.4±0.2, 27.1±0.2, or 33.2±0.2. In certain embodiments, the crystalline form exhibits an X-ray powder diffraction pattern comprising peaks at at least five of the following diffraction angles (2θ): 5.3±0.2, 10.6±0.2, 14.1±0.2, 15.3±0.2, 18.5±0.2, 19.4±0.2, 21.0±0.2, 24.4±0.2, 26.4±0.2, 27.1±0.2, or 33.2±0.2. In certain embodiments, the crystalline form exhibits an X-ray powder diffraction pattern comprising peaks at at least seven of the following diffraction angles (2θ): 5.3±0.2, 10.6±0.2, 14.1±0.2, 15.3±0.2, 18.5±0.2, 19.4±0.2, 21.0±0.2, 24.4±0.2, 26.4±0.2, 27.1±0.2, or 33.2±0.2.

In certain embodiments, the crystalline form is characterized by the following X-ray powder diffraction pattern expressed in terms of diffraction angle 2θ, inter-planar distances d, and relative intensity (expressed as a percentage with respect to the most intense peak):

| Angle [2θ] | d-spacing [Å] | Relative Intensity [%] |
|---|---|---|
| 5.3 | 16.6 | 20 |
| 9.4 | 9.4 | 6 |
| 10.6 | 8.3 | 39 |
| 13.2 | 6.7 | 5 |
| 14.1 | 6.3 | 27 |
| 15.3 | 5.8 | 21 |
| 16.4 | 5.4 | 5 |
| 18.5 | 4.8 | 10 |
| 19.4 | 4.6 | 21 |
| 20.2 | 4.4 | 6 |
| 21.0 | 4.2 | 21 |
| 24.4 | 3.6 | 22 |
| 26.4 | 3.4 | 98 |
| 27.1 | 3.3 | 100 |
| 33.2 | 2.7 | 22 |

### I. Form I Crystalline Disodium Salt of Compound I

One aspect of the invention provides Form I crystalline disodium salt of compound I. The disodium salt of compound I has the chemical formula:

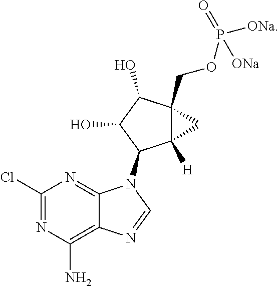

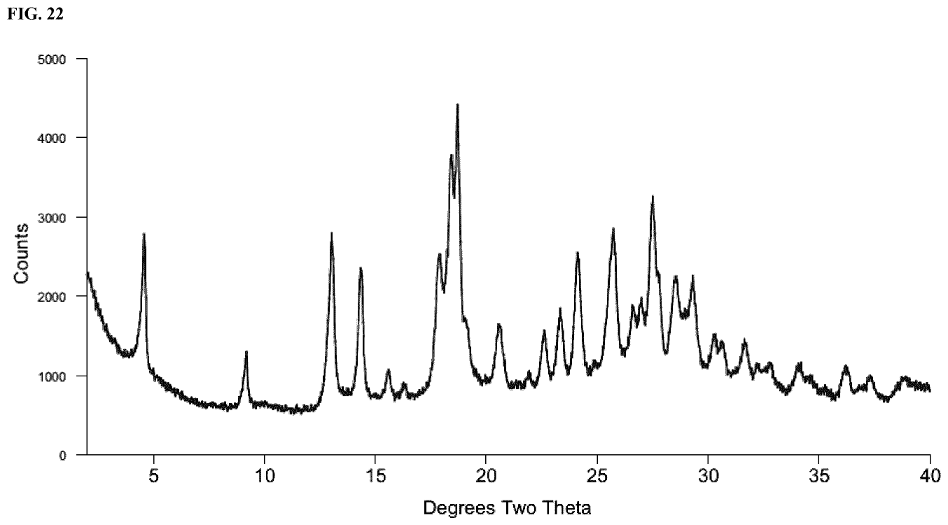

The Form I crystalline disodium salt of compound I may be characterized according to the X-ray powder diffractogram provided in FIG. 22. Accordingly, in certain embodiments, the Form I crystalline disodium salt of compound I may be characterized as having an X-ray powder diffractogram substantially the same as the X-ray powder diffractogram provided in FIG. 22. In certain embodiments, the crystalline form exhibits an X-ray powder diffraction pattern comprising peaks at one or more of the following diffraction angles (2θ): 4.6±0.2, 9.2±0.2, 13.1±0.2, 14.4±0.2, 17.9±0.2, 18.4±0.2, 18.7±0.2, 20.6±0.2, 22.6±0.2, 23.3±0.2, 24.1±0.2, 25.7±0.2, 27.0±0.2, 27.5±0.2, 28.5±0.2, or 29.3±0.2. In certain embodiments, the crystalline form exhibits an X-ray powder diffraction pattern comprising peaks at at least five of the following diffraction angles (2θ): 4.6±0.2, 9.2±0.2, 13.1±0.2, 14.4±0.2, 17.9±0.2, 18.4±0.2, 18.7±0.2, 20.6±0.2, 22.6±0.2, 23.3±0.2, 24.1±0.2, 25.7±0.2, 27.0±0.2, 27.5±0.2, 28.5±0.2, or 29.3±0.2. In certain embodiments, the crystalline form exhibits an X-ray powder diffraction pattern comprising peaks at at least seven of the following diffraction angles (2θ): 4.6±0.2, 9.2±0.2, 13.1±0.2, 14.4±0.2, 17.9±0.2, 18.4±0.2, 18.7±0.2, 20.6±0.2, 22.6±0.2, 23.3±0.2, 24.1±0.2, 25.7±0.2, 27.0±0.2, 27.5±0.2, 28.5±0.2, or 29.3±0.2.

In certain embodiments, the crystalline form is characterized by the following X-ray powder diffraction pattern expressed in terms of diffraction angle 2θ, inter-planar distances d, and relative intensity (expressed as a percentage with respect to the most intense peak):

| Angle [2θ] | d-spacing [Å] | Relative Intensity [%] |
|---|---|---|
| 4.6 | 19.2 | 34 |
| 9.2 | 9.6 | 15 |
| 13.1 | 6.8 | 48 |
| 14.4 | 6.2 | 36 |
| 15.6 | 5.7 | 5 |
| 17.9 | 5.0 | 62 |
| 18.4 | 4.8 | 100 |
| 18.7 | 4.7 | 35 |
| 20.6 | 4.3 | 16 |
| 22.6 | 3.9 | 10 |
| 23.3 | 3.8 | 15 |
| 24.1 | 3.7 | 30 |
| 25.7 | 3.5 | 42 |
| 26.6 | 3.3 | 9 |
| 27.0 | 3.3 | 16 |
| 27.5 | 3.2 | 51 |
| 28.5 | 3.1 | 27 |
| 29.3 | 3.0 | 27 |
| 30.2 | 3.0 | 10 |
| 31.6 | 2.8 | 7 |
| 32.8 | 2.7 | 7 |
| 34.0 | 2.6 | 9 |
| 36.2 | 2.5 | 8 |
| 37.3 | 2.4 | 5 |
| 38.7 | 2.3 | 4 |

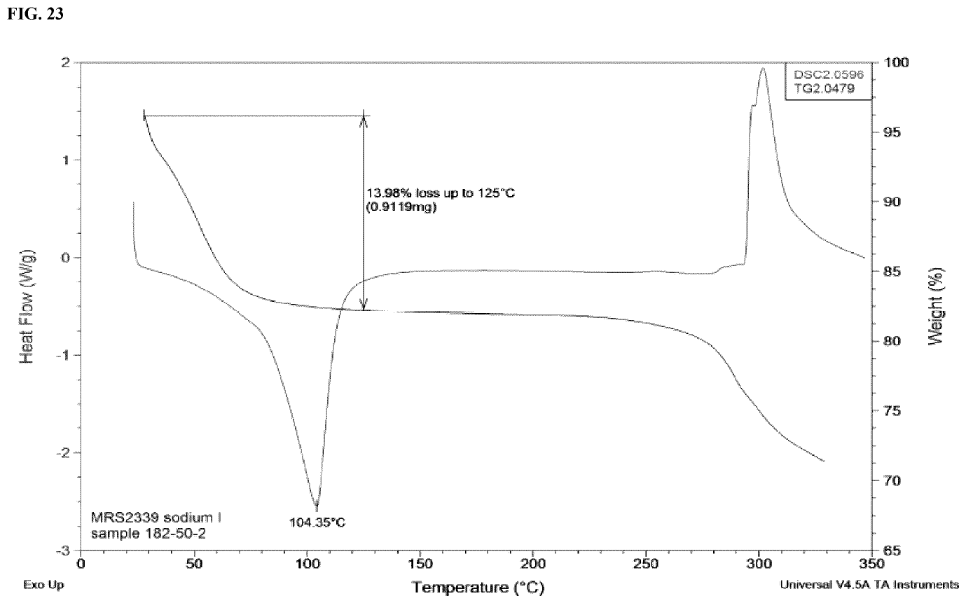

In certain embodiments, the Form I crystalline monosodium salt of compound I may be characterized according to differential scanning calorimetry and/or a thermogravimetric analysis. In certain embodiments, the Form I crystalline monosodium salt of compound I may be characterized as having a differential scanning calorimetry curve and/or a thermogravimetric analysis curve substantially the same as that provided in FIG. 23.

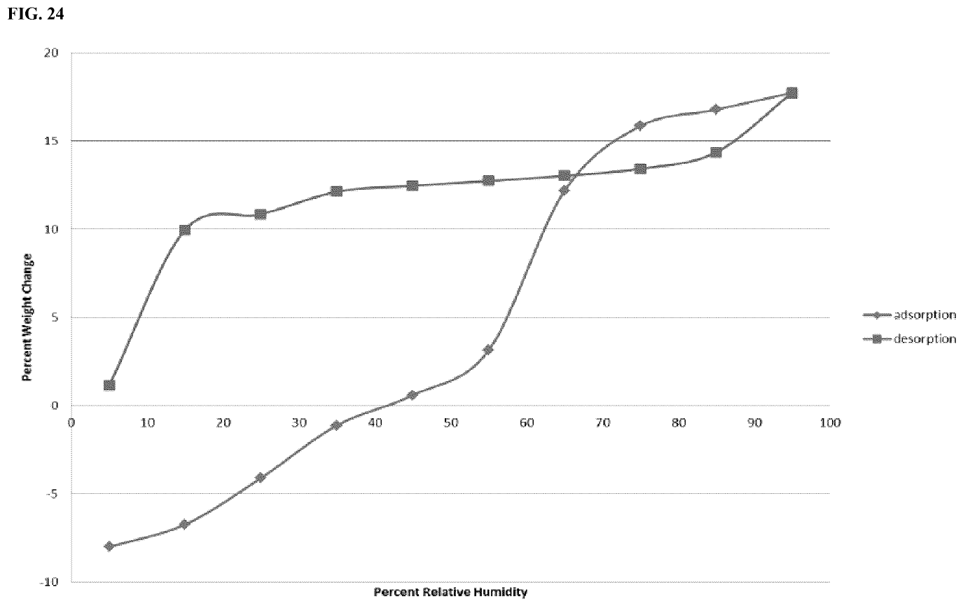

In certain embodiments, the Form I crystalline monosodium salt of compound I may be characterized according to dynamic vapor sorption. In certain embodiments, the Form I crystalline monosodium salt of compound I may be characterized according to dynamic vapor sorption curve that is substantially the same as that provided in FIG. 24.

### J. Form J Crystalline Monosodium Salt of Compound I

One aspect of the invention provides Form J crystalline monosodium salt of compound I. The monosodium salt of compound I has the chemical formula:

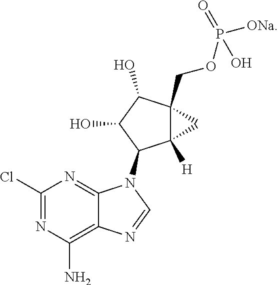

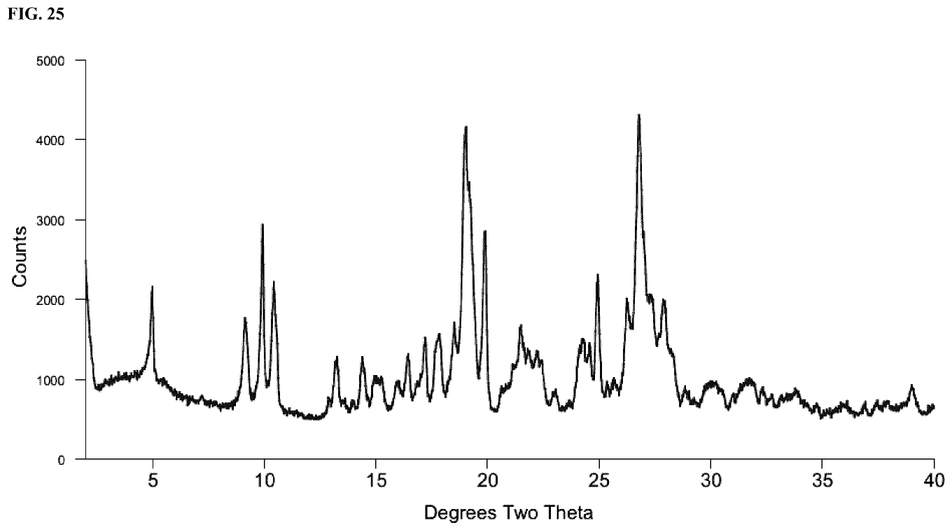

The Form J crystalline monosodium salt of compound I may be characterized according to the X-ray powder diffractogram provided in FIG. 25. Accordingly, in certain embodiments, the Form J crystalline monosodium salt of compound I may be characterized as having an X-ray powder diffractogram substantially the same as the X-ray powder diffractogram provided in FIG. 25. In certain embodiments, the crystalline form exhibits an X-ray powder diffraction pattern comprising peaks at one or more of the following diffraction angles (2θ): 5.0±0.2, 9.2±0.2, 9.9±0.2, 10.4±0.2, 13.2±0.2, 14.4±0.2, 15.1±0.2, 17.2±0.2, 17.8±0.2, 18.5±0.2, 19.0±0.2, 19.2±0.2, 19.9±0.2, 21.5±0.2, 21.8±0.2, 22.2±0.2, 24.1±0.2, 26.3±0.2, 26.8±0.2, 27.0±0.2, 27.4±0.2, 27.9±0.2, 30.0±0.2, or 31.6±0.2. In certain embodiments, the crystalline form exhibits an X-ray powder diffraction pattern comprising peaks at at least five of the following diffraction angles (2θ): 5.0±0.2, 9.2±0.2, 9.9±0.2, 10.4±0.2, 13.2±0.2, 14.4±0.2, 15.1±0.2, 17.2±0.2, 17.8±0.2, 18.5±0.2, 19.0±0.2, 19.2±0.2, 19.9±0.2, 21.5±0.2, 21.8±0.2, 22.2±0.2, 24.1±0.2, 26.3±0.2, 26.8±0.2, 27.0±0.2, 27.4±0.2, 27.9±0.2, 30.0±0.2, or 31.6±0.2. In certain embodiments, the crystalline form exhibits an X-ray powder diffraction pattern comprising peaks at at least seven of the following diffraction angles (2θ): 5.0±0.2, 9.2±0.2, 9.9±0.2, 10.4±0.2, 13.2±0.2, 14.4±0.2, 15.1±0.2, 17.2±0.2, 17.8±0.2, 18.5±0.2, 19.0±0.2, 19.2±0.2, 19.9±0.2, 21.5±0.2, 21.8±0.2, 22.2±0.2, 24.1±0.2, 26.3±0.2, 26.8±0.2, 27.0±0.2, 27.4±0.2, 27.9±0.2, 30.0±0.2, or 31.6±0.2.

In certain embodiments, the crystalline form is characterized by the following X-ray powder diffraction pattern expressed in terms of diffraction angle 2θ, inter-planar distances d, and relative intensity (expressed as a percentage with respect to the most intense peak):

| Angle [2θ] | d-spacing [Å] | Relative Intensity [%] |
|---|---|---|
| 5.0 | 17.7 | 64 |
| 9.2 | 9.7 | 46 |
| 9.9 | 8.9 | 58 |
| 10.4 | 8.5 | 70 |
| 13.2 | 6.7 | 28 |
| 14.4 | 6.1 | 14 |
| 15.1 | 5.9 | 14 |
| 16.4 | 5.4 | 12 |
| 17.2 | 5.1 | 17 |
| 17.8 | 5.0 | 22 |
| 18.5 | 4.8 | 22 |
| 19.0 | 4.7 | 100 |
| 19.2 | 4.6 | 75 |
| 19.9 | 4.5 | 51 |
| 21.5 | 4.1 | 46 |
| 21.8 | 4.1 | 17 |
| 22.2 | 4.0 | 25 |
| 24.1 | 3.7 | 48 |
| 24.6 | 3.6 | 7 |
| 24.9 | 3.6 | 32 |
| 25.7 | 3.5 | 10 |
| 26.3 | 3.4 | 62 |
| 26.8 | 3.3 | 78 |
| 27.0 | 3.3 | 47 |
| 27.4 | 3.3 | 73 |
| 27.9 | 3.2 | 66 |
| 30.0 | 3.0 | 12 |
| 31.6 | 2.8 | 22 |
| 33.8 | 2.6 | 14 |
| 37.9 | 2.4 | 8 |
| 39.0 | 2.3 | 14 |

### K. Form N Crystalline Monosodium Salt of Compound I

One aspect of the invention provides Form N crystalline monosodium salt of compound I. The monosodium salt of compound I has the chemical formula:

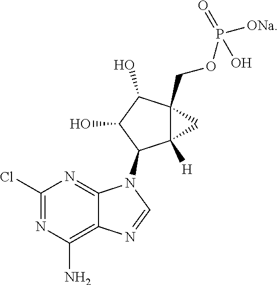

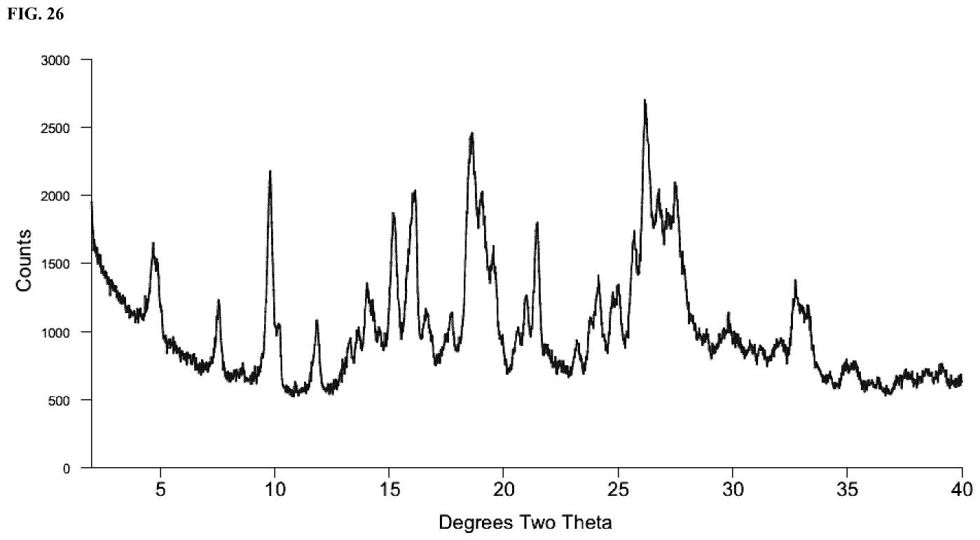

Form N crystalline monosodium salt of compound I may be characterized according to the X-ray powder diffractogram provided in FIG. 26. Accordingly, in certain embodiments, the Form N crystalline monosodium salt of compound I may be characterized as having an X-ray powder diffractogram substantially the same as the X-ray powder diffractogram provided in FIG. 26. In certain embodiments, the crystalline form exhibits an X-ray powder diffraction pattern comprising peaks at one or more of the following diffraction angles (2θ): 4.7±0.2, 7.6±0.2, 9.8±0.2, 10.2±0.2, 14.0±0.2, 15.2±0.2, 16.1±0.2, 18.5±0.2, 19.6±0.2, 21.5±0.2, 24.1±0.2, 24.9±0.2, 25.6±0.2, 26.2±0.2, 26.7±0.2, 27.5±0.2, or 32.8±0.2. In certain embodiments, the crystalline form exhibits an X-ray powder diffraction pattern comprising peaks at at least five of the following diffraction angles (2θ): 4.7±0.2, 7.6±0.2, 9.8±0.2, 10.2±0.2, 14.0±0.2, 15.2±0.2, 16.1±0.2, 18.5±0.2, 19.6±0.2, 21.5±0.2, 24.1±0.2, 24.9±0.2, 25.6±0.2, 26.2±0.2, 26.7±0.2, 27.5±0.2, or 32.8±0.2. In certain embodiments, the crystalline form exhibits an X-ray powder diffraction pattern comprising peaks at at least seven of the following diffraction angles (2θ): 4.7±0.2, 7.6±0.2, 9.8±0.2, 10.2±0.2, 14.0±0.2, 15.2±0.2, 16.1±0.2, 18.5±0.2, 19.6±0.2, 21.5±0.2, 24.1±0.2, 24.9±0.2, 25.6±0.2, 26.2±0.2, 26.7±0.2, 27.5±0.2, or 32.8±0.2.

In certain embodiments, the crystalline form is characterized by the following X-ray powder diffraction pattern expressed in terms of diffraction angle 2θ, inter-planar distances d, and relative intensity (expressed as a percentage with respect to the most intense peak):

| Angle [2θ] | d-spacing [Å] | Relative Intensity [%] |
|---|---|---|
| 4.7 | 18.7 | 31 |
| 7.6 | 11.6 | 13 |
| 9.8 | 9.0 | 37 |
| 10.2 | 8.7 | 30 |
| 11.8 | 7.5 | 10 |
| 13.3 | 6.7 | 5 |
| 13.6 | 6.5 | 6 |
| 14.0 | 6.3 | 23 |
| 15.2 | 5.8 | 30 |
| 16.1 | 5.5 | 46 |
| 16.6 | 5.3 | 5 |
| 17.7 | 5.0 | 3 |
| 18.5 | 4.8 | 100 |
| 19.6 | 4.5 | 21 |
| 20.6 | 4.3 | 4 |
| 21.0 | 4.2 | 12 |
| 21.5 | 4.1 | 27 |
| 24.1 | 3.7 | 20 |
| 24.9 | 3.6 | 18 |
| 25.6 | 3.5 | 32 |
| 26.2 | 3.4 | 58 |
| 26.7 | 3.3 | 71 |
| 27.5 | 3.2 | 35 |
| 32.0 | 2.8 | 5 |
| 32.8 | 2.7 | 41 |
| 35.1 | 2.6 | 7 |

### III. Methods for Making a Crystalline Hydrate of Compound I

Another aspect of the invention provides a method of preparing a crystalline hydrate of compound I, comprising the steps of:
(a) admixing compound I, methanol, and water to form a mixture, and heating said mixture to a temperature greater than 30° C. to form a heated mixture; and
(b) cooling said heated mixture to thereby form a precipitate that is the crystalline hydrate of compound I;
   wherein compound I is represented by:

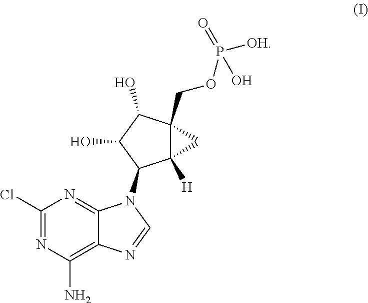 [SMILES: Nc1nc(Cl)nc2c1ncn2[C@H]1[C@H](O)[C@H](O)[C@]2(COP(=O)(O)O)C[C@H]12]

The method may be characterized by additional features, such as the volume ratio of methanol to water used in step (a), temperatures used in the method, and other features. For example, in certain embodiments, the volume ratio of methanol to water used in step (a) is about 10:1. In certain embodiments, the volume ratio of methanol to water used in step (a) is from about 8:1 to about 11:1.

In certain embodiments, in step (a) heating said mixture comprises heating said mixture to a temperature in the range of from about 55° C. to about 65° C. In certain embodiments, in step (a) heating said mixture comprises heating said mixture to a temperature of about 65° C. In certain embodiments, in step (a) heating said mixture comprises heating said mixture to a temperature of 65° C.

In certain embodiments, cooling said heated mixture in step (b) comprises cooling said mixture to a temperature in the range of about 15° C. to about 25° C. In certain embodiments, cooling said heated mixture in step (b) comprises cooling said mixture to a temperature in the range of 15° C. to 25° C. In certain embodiments, cooling said heated mixture in step (b) comprises cooling said mixture to a temperature in the range of about 18° C. to about 20° C. In certain embodiments, cooling said heated mixture in step (b) comprises cooling said mixture to a temperature in the range of 18° C. to 20° C. In certain embodiments, cooling said heated mixture in step (b) comprises cooling said mixture to a temperature of 18° C.

In certain embodiments, the method further comprises the step of isolating the prepreciptate formed in step (b).

Another aspect of the invention provides a crystalline hydrate of compound I formed according to the method described herein above.

In certain embodiments, the crystalline hydrate of compound I formed by the method is further characterized according to one or more features described above in Section I for Form B Crystalline Hydrate of Compound I. For example, in certain embodiments, the crystalline form exhibits an X-ray powder diffraction pattern comprising peaks at the following diffraction angles (2θ): 8.9±0.2, 15.8±0.2, 21.3±0.2, 24.7±0.2, 26.4±0.2, 27.2±0.2, and 33.0±0.2. In certain embodiments, the X-ray powder diffraction pattern further comprises a peak at the following diffraction angle (2θ): 10.1±0.2. In certain embodiments, the X-ray powder diffraction pattern further comprises a peak at the following diffraction angle (2θ): 12.9±0.2. In certain embodiments, the X-ray powder diffraction pattern further comprises a peak at the following diffraction angle (2θ): 18.1±0.2. In certain embodiments, the X-ray powder diffraction pattern further comprises a peak at the following diffraction angle (2θ): 20.5±0.2. In certain embodiments, the X-ray powder diffraction pattern further comprises a peak at the following diffraction angle (2θ): 23.8±0.2. In certain embodiments, the X-ray powder diffraction pattern further comprises a peak at the following diffraction angle (2θ): 32.2±0.2.

In certain embodiments, the relative intensity of the peak at said diffraction angles (2θ) is at least 30%. In certain embodiments, the relative intensity of the peak at said diffraction angles (2θ) is at least 25%. In certain embodiments, the relative intensity of the peak at said diffraction angles (2θ) is at least 20%. In certain embodiments, the relative intensity of the peak at said diffraction angles (2θ) is at least 15%. In certain embodiments, the relative intensity of the peak at said diffraction angles (2θ) is at least 10%.

### IV. Therapeutic Applications

The compounds described herein provide therapeutic benefits to subjects suffering from medical disorders, including cardiac disorders. Accordingly, provided herein is a method for treating a cardiac disorder, comprising administering to a subject in need thereof a therapeutically effective amount of a compound described herein, to treat the cardiac disorder. The compounds may be part of a pharmaceutical composition. In certain embodiments, the compounds are dissolved into a liquid pharmaceutical composition. The liquid pharmaceutical composition may be administered to the patient, such as via intravenous administration.

Another aspect of the invention provides a method for improving cardiac contractile performance in a subject, wherein the method comprises administering to a subject in need thereof an effective amount of a compound described herein to improve cardiac contractile performance. The compounds may be part of a pharmaceutical composition. In certain embodiments, the compounds are dissolved into a liquid pharmaceutical composition. The liquid pharmaceutical composition may be administered to the patient, such as via intravenous administration.

Another aspect of the invention provides a method for improving cardiac function in a subject, wherein the method comprises administering to a subject in need thereof an effective amount of a compound described herein to improve cardiac function. The compounds may be part of a pharmaceutical composition. In certain embodiments, the compounds are dissolved into a liquid pharmaceutical composition. The liquid pharmaceutical composition may be administered to the patient, such as via intravenous administration.

In certain embodiments, the improved cardiac function is characterized by one or more of improved ability of the heart to relax, favorable remodeling in a subject with heart failure, decreased fibrosis, decreased hypertrophy of cardiac myocytes, or improved calcium handling in a myocyte in a heart failure subject.

Another aspect of the invention provides a method of stimulating the activity of a cardiac P2X receptor in a subject, comprising administering to the subject in need thereof an effective amount of a compound described herein to stimulate the activity of said cardiac P2X receptor. The compounds may be part of a pharmaceutical composition. In certain embodiments, the compounds are dissolved into a liquid pharmaceutical composition. The liquid pharmaceutical composition may be administered to the patient, such as via intravenous administration.

In certain embodiments, the subject has a cardiac disorder. In certain embodiments, the cardiac disorder is heart failure, cardiac hypertrophy, ischemic cardiomyopathy, non-ischemic cardiomyopathy, or adverse remodeling and injury following ischemia/reperfusion injury. In certain embodiments, the cardiac disorder is heart failure. In certain embodiments, the cardiac disorder is cardiac hypertrophy. In certain embodiments, the cardiac disorder is ischemic cardiomyopathy. In certain embodiments, the cardiac disorder is non-ischemic cardiomyopathy. In certain embodiments, the cardiac disorder is adverse remodeling and injury following ischemia/reperfusion injury. In certain embodiments, the subject has more than one of any of the foregoing cardiac disorders.

In certain embodiments, the cardiac disorder is heart failure. In certain embodiments, the heart failure is one or more of systolic heart failure or diastolic heart failure. In certain embodiments, the heart failure is systolic heart failure. In certain embodiments, the heart failure is diastolic heart failure.

In certain embodiments, the subject is a human. In certain embodiments, the subject is an adult human. In certain embodiments, the subject is a pediatric human. In certain embodiments, the subject is a geriatric human.

Another aspect of the invention provides for the use of a compound described herein in the manufacture of a medicament. In certain embodiments, the medicament is for treating a disease or condition described herein, such as heart failure.

Another aspect of the invention provides for the use of compound described herein for treating a disease or condition, such as a disease or condition described herein, such as heart failure.

Another aspect of this invention is a kit comprising a compound described herein. In certain embodiments, the kit further comprises instructions, such as instructions for treating a disease or condition described herein. The compounds may be part of a pharmaceutical composition. In certain embodiments, the compounds are dissolved into a liquid pharmaceutical composition. The liquid pharmaceutical composition may be administered to the patient, such as via intravenous administration.

### V. Administration Aspects

The compounds described herein of the present invention are desirably formulated into a liquid pharmaceutical composition that is administered by intravenous administration. Actual dosage amount of the liquid pharmaceutical composition of this invention may be varied so as to provide an amount of the active ingredient which is effective to achieve the desired therapeutic response for a particular patient, composition, and mode of administration, without being toxic to the patient.

The selected dosage level will depend upon a variety of factors including the activity of the particular aqueous injectable formulations of the present disclosure employed, the route of administration, the time of administration, the rate of excretion or metabolism of the particular compound being employed, the rate and extent of absorption, the duration of the treatment, other drugs, compounds and/or materials used in combination with the particular liquid pharmaceutical composition employed, the age, sex, weight, condition, general health and prior medical history of the patient being treated, and like factors well known in the medical arts.

A physician or veterinarian having ordinary skill in the art can readily determine and prescribe the effective amount of the liquid pharmaceutical composition required. For example, the physician or veterinarian could start doses of the aqueous injectable formulations of the invention at levels lower than that required in order to achieve the desired therapeutic effect and gradually increase the dosage until the desired effect is achieved.

In general, a suitable daily dose of liquid pharmaceutical composition of the invention will be that amount of the liquid pharmaceutical composition which is the lowest dose effective to produce a therapeutic effect. Such an effective dose will generally depend upon the factors described above.

If desired, the effective daily dose of the aqueous injectable formulations may be administered as two, three, four, five, six or more sub-doses administered separately at appropriate intervals throughout the day, optionally, in unit dosage forms.

Another aspect of this invention is a kit comprising a therapeutically effective amount of a compound described herein and a pharmaceutically acceptable carrier, vehicle or diluent. In certain embodiments, the kit further comprises instructions, such as instructions for treating a disease described herein.

### VI. Methods for Making Pharmaceutical Compositions

Another aspect of the invention provides a method of preparing a pharmaceutical composition, wherein the method comprises admixing (i) a crystalline hydrate compound described herein or a salt compound described and (ii) a pharmaceutically acceptable carrier. In certain embodiments, the method comprises admixing (i) a crystalline hydrate compound described herein and (ii) a pharmaceutically acceptable carrier. In certain embodiments, the pharmaceutically acceptable carrier comprises water.

Another aspect of the invention provides a pharmaceutical composition made according to a method described herein.

### VII. Pharmaceutical Compositions and Dosing Considerations

As indicated above, the invention provides pharmaceutical compositions, which may comprise a therapeutically-effective amount of one or more of the compounds described above, formulated together with one or more pharmaceutically acceptable carriers (additives) and/or diluents. The pharmaceutical compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally. In certain embodiments, the invention provides a compound described herein and a pharmaceutically acceptable carrier.

The phrase “therapeutically effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

The invention further provides a unit dosage form comprising a compound described herein in a therapeutically effective amount for the treatment of a disease or condition described herein.

### EXAMPLES

The invention now being generally described, will be more readily understood by reference to the following examples, which are included merely for purposes of illustration of certain aspects and embodiments of the present invention, and are not intended to limit the invention. Starting materials described herein can be obtained from commercial sources or may be readily prepared from commercially available materials using transformations known to those of skill in the art.

### Example 1—Preparation of Form a Crystalline Hydrate of Compound I

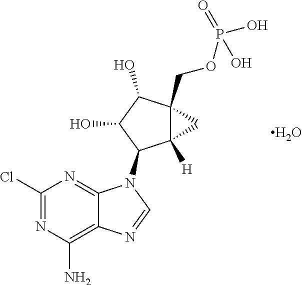 [SMILES: Nc1nc(Cl)nc2c1ncn2[C@H]1[C@H](O)[C@H](O)[C@]2(COP(=O)(O)O)C[C@H]12 ; O]

Dowex-50 (270 g) was washed with 3×800 mL portions of 1:1 MeOH-water by filtration. A 5 L reactor with mechanical stirring and a reflux condenser was charged with 95 g (0.175 mol) of (1'S,2′R,3'S,4′R,5'S)-4-(6-amino-2-chloro-9H-purin-9-yl)-1-[(di-tert-butylphosphate)methyl]bicyclo[3.1.0]hexane-2,3-(0-isopropylidene) and 3 L of 1:1 MeOH-deioniozed water. With stirring, the washed Dowex-50 resin was added to the reaction mixture over about 5 min. The resulting reaction mixture was heated to an internal temperature of 70-80° C. for 45 min then checked by HPLC. Then, the reaction solution was decanted hot. The remaining resin and ˜100 mL of reaction solution was re-suspended in 900 mL of 1:1 MeOH-water, heated to a temperature of 80° C. and then the solution filtered hot. The process was repeated with 1 L of water. The pooled filtrates were concentrated on a rotary evaporator at 45° C. under high vacuum giving 56 g of crude product (82%) as a white solid. The crude product was suspended in 2 L of MeOH and refluxed (70° C.) for 1 h, cooled to 0° C. and filtered. The filter cake was washed with 2×20 mL of MeOH, and then air dried overnight, and then subjected to vacuum removal of trace volatiles to provide 52.3 g (76%) of the title compound, namely Form A Crystalline Hydrate of Compound I.

### Example 2—Characterization of Form A Crystalline Hydrate of Compound I

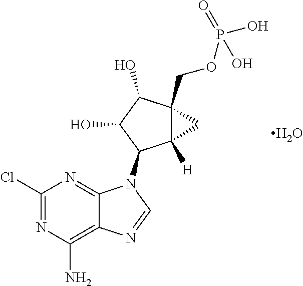 [SMILES: Nc1nc(Cl)nc2c1ncn2[C@H]1[C@H](O)[C@H](O)[C@]2(COP(=O)(O)O)C[C@H]12 ; O]

Form A crystalline hydrate of compound I was analyzed by X-ray powder diffraction, thermogravimetric analysis, dynamic vapor sorption (to analyze hygroscopicity), and Karl Fischer titration.

An X-ray powder diffractogram of one batch of the title compound is provided in FIG. 1. Tabulated characteristics of the X-ray powder diffractogram in FIG. 1 are provided in the following table, which lists diffraction angle 2θ, inter-planar distances d, and relative intensity (expressed as a percentage with respect to the most intense peak):

### X-Ray Powder Diffractogram Data

| Angle [2θ] | d-spacing [Å] | Relative Intensity [%] |
|---|---|---|
| 5.2 | 17.1 | 18 |
| 6.5 | 13.6 | 18 |
| 9.5 | 9.3 | 26 |
| 10.3 | 8.6 | 52 |
| 11.2 | 7.9 | 7 |
| 12.5 | 7.1 | 8 |
| 13.1 | 6.8 | 6 |
| 13.8 | 6.4 | 73 |
| 13.9 | 6.4 | 24 |
| 15.4 | 5.7 | 14 |
| 16.2 | 5.5 | 21 |
| 16.9 | 5.2 | 10 |
| 18.1 | 4.9 | 26 |
| 18.7 | 4.7 | 41 |
| 19.0 | 4.7 | 78 |
| 19.6 | 4.5 | 40 |
| 20.7 | 4.3 | 53 |
| 21.8 | 4.1 | 54 |
| 22.7 | 3.9 | 11 |
| 23.2 | 3.8 | 14 |
| 23.5 | 3.8 | 45 |
| 24.7 | 3.6 | 11 |
| 24.9 | 3.6 | 11 |
| 26.0 | 3.4 | 9 |
| 26.2 | 3.4 | 16 |
| 26.7 | 3.3 | 13 |
| 27.3 | 3.3 | 100 |
| 28.3 | 3.1 | 22 |
| 28.7 | 3.1 | 13 |
| 31.0 | 2.9 | 13 |
| 32.9 | 2.7 | 28 |
| 33.4 | 2.7 | 11 |
| 34.6 | 2.6 | 5 |
| 35.3 | 2.5 | 4 |
| 35.6 | 2.5 | 14 |
| 37.4 | 2.4 | 15 |
| 37.7 | 2.4 | 10 |
| 38.2 | 2.4 | 10 |
| 38.5 | 2.3 | 5 |
| 39.2 | 2.3 | 3 |

A thermogravimetric analysis curve of the title compound is provided in FIG. 2.

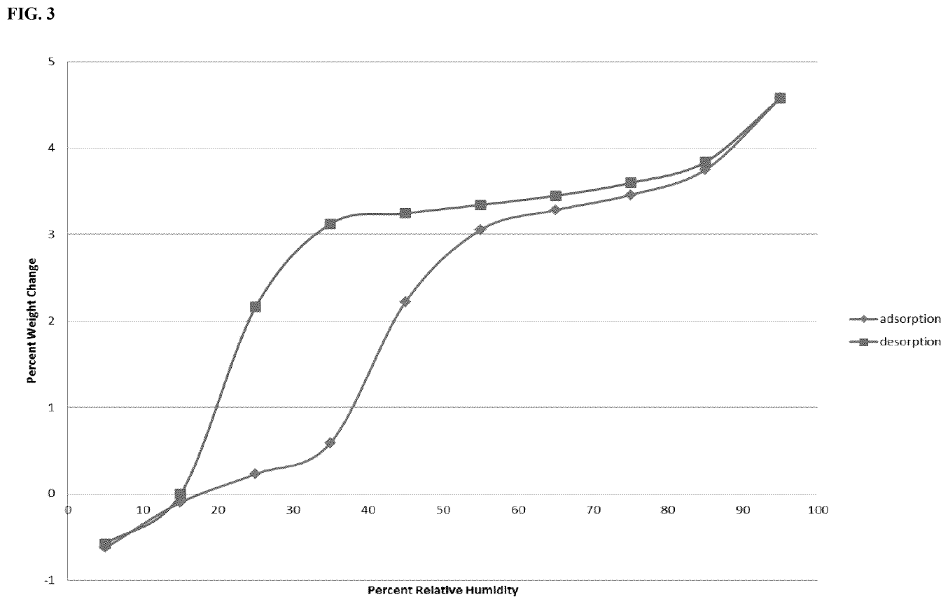

The title compound was analyzed for hygroscopicity by dynamic vapor sorption. Results of the analysis for hygroscopicity by dynamic vapor sorption are depicted in FIG. 3, where a 5.2100 increase in weight was observed during the absorption step of the experiment, and a 5.16% weight loss was observed during the desorption step of the experiment.

The title compound was analyzed for water content by Karl Fischer (KF) titration, and determined to have a water content of 4.67 wt 00 (corresponding to 0.98 moles of water).

A sample of Form A crystalline hydrate of Compound I was subjected to stability analysis for 1 week at a temperature of 25° C. at 60% relative humidity. At the end of the stability study, the compound sample was analyzed and determined to be Form B Crystalline Hydrate of Compound I. Hence, under the stability study conditions, Form A crystalline hydrate of Compound I converted to Form B crystalline hydrate of Compound I.

### Example 3—Preparation of Form B Crystalline Hydrate of Compound I

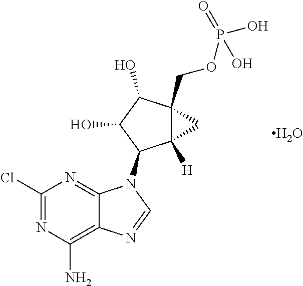 [SMILES: Nc1nc(Cl)nc2c1ncn2[C@H]1[C@H](O)[C@H](O)[C@]2(COP(=O)(O)O)C[C@H]12 ; O]

The title compound was prepared according to the following procedures.

### Part I

Compound ((3aR,3bR,4aS,5R,5aS)-5-(6-amino-2-chloro-9H-purin-9-yl)-2,2-dimethyltetrahydrocyclopropa[3,4]cyclopenta[1,2-d][1,3]dioxol-3b(3aH)-yl)methyl di-tert-butyl phosphate (24.21 kg) and solvent containing water and methanol were charged to a reactor vessel, and then a 2.0 M HCl solution was charged to the reactor vessel. Contents of the reactor vessel were then head to a temperature range of 62±5° C. and held at that temperature for about 4.75 hours with constant agitation. Then, contents of the reactor vessel were allowed to cool to room temperature with constant agitation; the end temperature of contents of the reactor vessel was 21.0° C.

Next, contents of the reactor vessel were cooled to a temperature of −5±5° C. and held at that temperature for 2 hr 15 mins with constant agitation. Then, contents of the reactor vessel were filtered using a Buchner Table-Top Funnel set up with 3-Neck Carboy under vacuum, and the filtrate was transferred to a clean container. The reactor vessel was charged with methanol, and then the contents in the reactor vessel were cooled to a target temperature range of −5±5° C. Then, contents of the reactor vessel were filtered using a Buchner Table-Top Funnel set up with 3-Neck Carboy under vacuum. Filter cake resulting from the filtrations was air dried for about 1.1 hours to provide crude ((1R,2R,3S,4R,5S)-4-(6-amino-2-chloro-9H-purin-9-yl)-2,3-dihydroxybicyclo[3.1.0]hexan-1-yl)methyl dihydrogen phosphate.

Next, the crude ((1R,2R,3S,4R,5S)-4-(6-amino-2-chloro-9H-purin-9-yl)-2,3-dihydroxybicyclo[3.1.0]hexan-1-yl)methyl dihydrogen phosphate was charged to a reactor vessel along with a 10:1 methanol/water solution. Temperature of the contents of the reactor vessel were heated to a temperature of 60±5° C. and then held at that temperature for 1 hr with constant agitation. Next, contents of the reactor vessel were cooled to room temperature with constant agitation, where the final temperature of contents in the reactor vessel were 19.0° C. Then, contents of the reactor vessel were cooled to a temperature range of −5±5° C. and then held at that temperature for 1 hr 5 min with constant agitation. Next, contents of the reactor vessel were filtered using a Buchner Table-Top Funnel set up with 3-Neck Carboy under vacuum, and the filtrate was transferred to a clean container. The reactor vessel was charged with methanol, and then the contents in the reactor vessel were cooled to a target temperature range of −5±5° C. Then, contents of the reactor vessel were filtered using a Buchner Table-Top Funnel set up with 3-Neck Carboy under vacuum. Filter cake resulting from the filtrations was air dried for about 47.5 hours to provide purified product.

### Part II

Next, the purified product from Part I was charged to a reactor vessel along with a 10:1 methanol/water solution. Temperature of the contents of the reactor vessel were heated to a temperature of 60±5° C. and then held at that temperature for about 1.1 hr with constant agitation. Next, contents of the reactor vessel were allowed to cool to room temperature with constant agitation, where the final temperature of contents in the reactor vessel were 19.0° C. Then, contents of the reactor vessel were cooled to a temperature range of −5±5° C. and then held at that temperature for 1 hr 5 min with constant agitation. Then, contents of the reactor vessel were held at 25° C. overnight, and then cooled to 0° C.

Next, contents of the reactor vessel were filtered using a Buchner Table-Top Funnel set up with 3-Neck Carboy under vacuum, and the filtrate was transferred to a clean container. The reactor vessel was charged with methanol, and then the contents in the reactor vessel were cooled to a target temperature range of −5±5° C. Then, contents of the reactor vessel were filtered using a Buchner Table-Top Funnel set up with 3-Neck Carboy under vacuum. Filter cake resulting from the filtrations was air dried for about 15 minutes to provide final purified acid product.

### Part III

Next, the final purified acid product from Part II was charged to a reactor vessel along with a 10:1 methanol/water solution. Contents of the reactor vessel were heated to a temperature of 60±5° C. and then held at that temperature for about 1.9 hr with constant agitation. Next, contents of the reactor vessel were cooled to room temperature with constant agitation, where the final temperature of contents in the reactor vessel was 18.0° C.

Next, contents of the reactor vessel were filtered using a Buchner Table-Top Funnel set up with 3-Neck Carboy under vacuum, and the filtrate was transferred to a clean container. The reactor vessel was charged with methanol, and then contents in the reactor vessel were cooled to a target temperature range of −5±5° C. Then, contents of the reactor vessel were filtered using a Buchner Table-Top Funnel set up with 3-Neck Carboy under vacuum. Filter cake resulting from the filtrations was air dried for about 15.75 hours to provide purified compound, which was charged to a reactor vessel along with a 10:1 methanol/water solution. Contents of the reactor vessel were then heated to a temperature of 60±5° C. and then held at that temperature for about 2.9 hr with constant agitation. Next, contents of the reactor vessel were cooled to room temperature with constant agitation, where the final temperature of contents in the reactor vessel were 21.0° C.

Next, contents of the reactor vessel were cooled to a temperature range of −5±5° C. and then contents of the reactor vessel were filtered using a Buchner Table-Top Funnel set up with 3-Neck Carboy under vacuum. Filter cake resulting from the filtration was air dried for about 20 minutes to provide the title compound, namely Form B Crystalline Hydrate of Compound I.

### Example 4—Alternative Preparation of Form B Crystalline Hydrate of Compound I

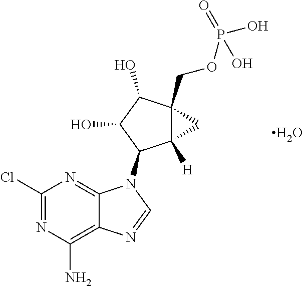 [SMILES: Nc1nc(Cl)nc2c1ncn2[C@H]1[C@H](O)[C@H](O)[C@]2(COP(=O)(O)O)C[C@H]12 ; O]

The title compound was prepared according to the following procedure: a 10 mg sample of Form A Crystalline Hydrate of Compound I was placed in a glass vial, and the uncapped vial was placed in an oven at 70° C. and 75% RH for 7 days, to thereby provide the title compound, namely Form B Crystalline Hydrate of Compound I.

### Example 5—Characterization of Form B Crystalline Hydrate of Compound I

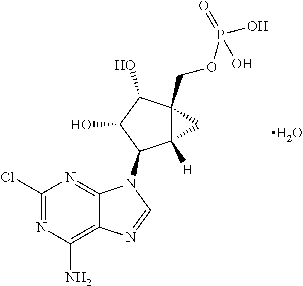 [SMILES: Nc1nc(Cl)nc2c1ncn2[C@H]1[C@H](O)[C@H](O)[C@]2(COP(=O)(O)O)C[C@H]12 ; O]

Form B crystalline hydrate of compound I was analyzed by X-ray powder diffraction, simultaneous thermogravimetric analysis and differential scanning calorimetry, differential scanning calorimetry, dynamic vapor sorption (to analyze hygroscopicity), and Karl Fischer titration.

An X-ray powder diffractogram of one batch of the title compound is provided in FIG. 4. Tabulated characteristics of the X-ray powder diffractogram in FIG. 4 are provided in the following table, which lists diffraction angle 2θ, inter-planar distances d, and relative intensity (expressed as a percentage with respect to the most intense peak):

### X-Ray Powder Diffractogram Data

| Angle [2θ] | d-spacing [Å] | Relative Intensity [%] |
|---|---|---|
| 5.1 | 17.3 | 30 |
| 8.9 | 9.9 | 66 |
| 9.8 | 9.0 | 28 |
| 10.1 | 8.7 | 49 |
| 12.9 | 6.8 | 15 |
| 14.7 | 6.0 | 54 |
| 15.1 | 5.8 | 2 |
| 15.8 | 5.6 | 21 |
| 16.4 | 5.4 | 8 |
| 17.0 | 5.2 | 18 |
| 18.1 | 4.9 | 30 |
| 18.4 | 4.8 | 22 |
| 18.8 | 4.7 | 48 |
| 19.2 | 4.6 | 16 |
| 19.4 | 4.6 | 21 |
| 19.7 | 4.5 | 30 |
| 20.5 | 4.3 | 14 |
| 21.3 | 4.2 | 58 |
| 22.1 | 4.0 | 13 |
| 22.4 | 4.0 | 11 |
| 22.9 | 3.9 | 11 |
| 23.8 | 3.7 | 15 |
| 24.7 | 3.6 | 58 |
| 25.4 | 3.5 | 14 |
| 26.0 | 3.4 | 13 |
| 26.4 | 3.4 | 100 |
| 27.2 | 3.3 | 41 |
| 27.8 | 3.2 | 21 |
| 30.5 | 2.9 | 7 |
| 30.9 | 2.9 | 7 |
| 31.8 | 2.8 | 8 |
| 32.2 | 2.8 | 20 |
| 33.0 | 2.7 | 24 |
| 33.5 | 2.7 | 14 |
| 36.6 | 2.5 | 7 |
| 37.7 | 2.4 | 13 |
| 38.9 | 2.3 | 21 |

The title compound was analyzed for water content by Karl Fischer (KF) titration, and determined to have a water content of 6.80 wt % (corresponding to 1.46 moles of water).

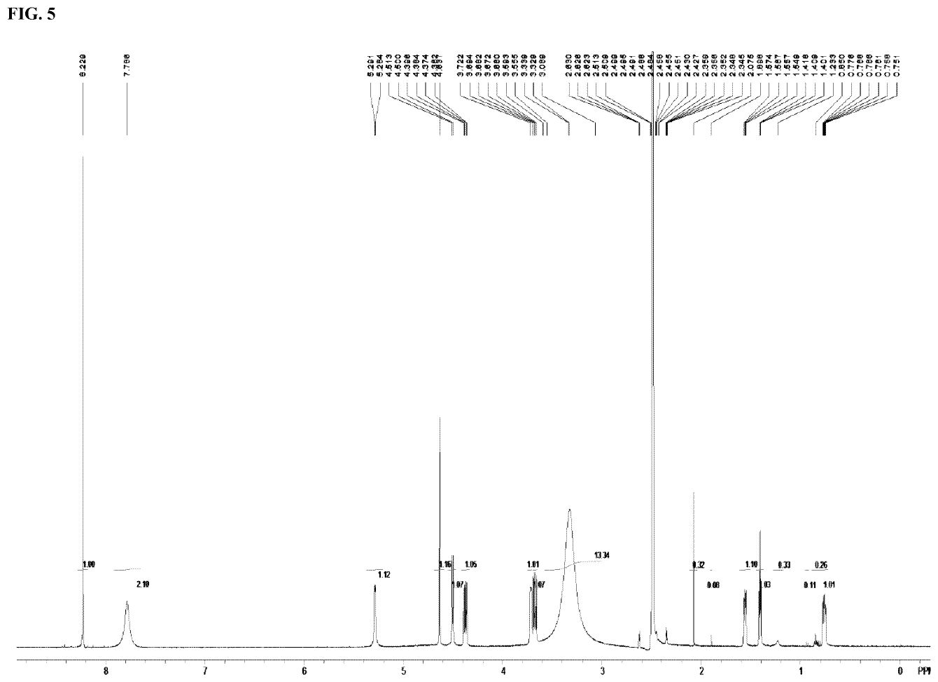

A sample of Form B crystalline hydrate of compound I was dissolved in deuterated solvent and then subjected to ¹H NMR analysis. The ¹H NMR spectrum I provided FIG. 5.

Stability of Form B crystalline hydrate of compound I to extended storage was evaluated by storing samples of Form B crystalline hydrate of compound I for twelve months at either 5° C. or 25° C., and then subjecting samples of the stored material to X-ray powder diffraction analytical analysis. Samples of Form B crystalline hydrate of compound I subjected to storage for twelve months at either 5° C. or 25° C. were determined to remain as Form B crystalline hydrate of compound I for the duration of the stability analysis experiment.

### Example 6—Preparation of Form A Crystalline Monosodium Salt of Compound I

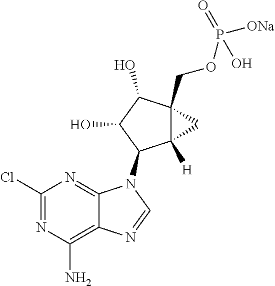

A slurry of ((1R,2R,3S,4R,5S)-4-(6-amino-2-chloro-9H-purin-9-yl)-2,3-dihydroxybicyclo[3.1.0]hexan-1-yl)methyl dihydrogen phosphate, water, and sodium hydroxide was prepared at room temperature, then the slurry was cooled to 5° C. to form a turbid mixture. The turbid mixture was then evaporated at room temperature to provide the title compound as a crystalline solid. An X-ray powder diffractogram of the title compound is provided in FIG. 6. Tabulated characteristics of the X-ray powder diffractogram in FIG. 6 are provided in the following table, which lists diffraction angle 2θ, inter-planar distances d, and relative intensity (expressed as a percentage with respect to the most intense peak):

### X-Ray Powder Diffractogram Data

| Angle [2θ] | d-spacing [Å] | Relative Intensity [%] |
|---|---|---|
| 5.2 | 17.0 | 12 |
| 5.7 | 15.6 | 11 |
| 10.4 | 8.5 | 44 |
| 14.3 | 6.2 | 5 |
| 19.5 | 4.6 | 33 |
| 21.2 | 4.2 | 4 |
| 26.6 | 3.3 | 100 |
| 32.9 | 2.7 | 12 |

### Example 7—Preparation of Form B Crystalline Disodium Salt of Compound I

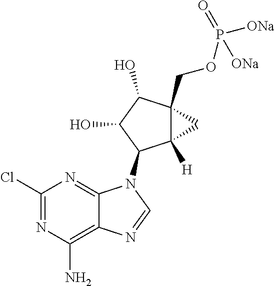

A slurry of ((1R,2R,3S,4R,5S)-4-(6-amino-2-chloro-9H-purin-9-yl)-2,3-dihydroxybicyclo[3.1.0]hexan-1-yl)methyl dihydrogen phosphate, acetone, and sodium hydroxide was prepared and held at 50° C. for one day. Then, the title compound was isolated as a crystalline solid. An X-ray powder diffractogram of the title compound is provided in FIG. 7. Tabulated characteristics of the X-ray powder diffractogram in FIG. 7 are provided in the following table, which lists diffraction angle 2θ, along with inter-planar distances d:

### X-Ray Powder Diffractogram Data

| Angle [2θ] | d-spacing [Å] |
|---|---|
| 5.4 | 16.3 |
| 8.8 | 10.0 |
| 9.8 | 9.0 |
| 10.2 | 8.7 |
| 11.1 | 8.0 |
| 13.2 | 6.7 |
| 13.8 | 6.4 |
| 15.0 | 5.9 |
| 18.1 | 4.9 |
| 19.3 | 4.6 |
| 19.9 | 4.5 |
| 20.3 | 4.4 |
| 24.4 | 3.6 |
| 26.7 | 3.3 |
| 27.6 | 3.2 |
| 31.8 | 2.8 |

### Example 8—Preparation of Form C Crystalline Monosodium Salt of Compound I

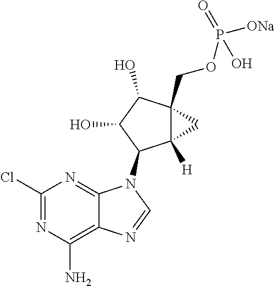

A slurry of ((1R,2R,3S,4R,5S)-4-(6-amino-2-chloro-9H-purin-9-yl)-2,3-dihydroxybicyclo[3.1.0]hexan-1-yl)methyl dihydrogen phosphate, acetone, and sodium hydroxide was prepared and held at 50° C. for two days. Then, the title compound was isolated as a crystalline solid. An X-ray powder diffractogram of the title compound is provided in FIG. 8. Tabulated characteristics of the X-ray powder diffractogram in FIG. 8 are provided in the following table, which lists diffraction angle 2θ, inter-planar distances d, and relative intensity (expressed as a percentage with respect to the most intense peak):

### X-Ray Powder Diffractogram Data

| Angle [2θ] | d-spacing [Å] | Relative Intensity [%] |
|---|---|---|
| 5.0 | 17.8 | 23 |
| 9.2 | 9.6 | 11 |
| 10.0 | 8.9 | 39 |
| 10.3 | 8.6 | 24 |
| 13.3 | 6.6 | 23 |
| 14.0 | 6.3 | 15 |
| 14.6 | 6.1 | 5 |
| 15.2 | 5.8 | 25 |
| 15.9 | 5.6 | 12 |
| 16.1 | 5.5 | 7 |
| 16.9 | 5.2 | 15 |
| 17.8 | 5.0 | 40 |
| 18.5 | 4.8 | 10 |
| 18.9 | 4.7 | 100 |
| 19.2 | 4.6 | 52 |
| 20.0 | 4.4 | 26 |
| 20.7 | 4.3 | 6 |
| 21.6 | 4.1 | 38 |
| 21.9 | 4.1 | 27 |
| 22.5 | 3.9 | 8 |
| 23.8 | 3.7 | 14 |
| 24.1 | 3.7 | 10 |
| 24.4 | 3.6 | 15 |
| 24.9 | 3.6 | 4 |
| 25.1 | 3.5 | 7 |
| 25.6 | 3.5 | 7 |
| 26.0 | 3.4 | 8 |
| 26.3 | 3.4 | 37 |
| 26.9 | 3.3 | 94 |
| 27.2 | 3.3 | 45 |
| 28.0 | 3.2 | 35 |
| 28.4 | 3.1 | 16 |
| 30.4 | 2.9 | 22 |
| 31.9 | 2.8 | 16 |
| 32.9 | 2.7 | 19 |
| 34.3 | 2.6 | 13 |
| 36.3 | 2.5 | 4 |
| 37.9 | 2.4 | 5 |
| 38.2 | 2.4 | 10 |

Results of differential scanning calorimetry and a thermogravimetric analysis are provided in FIG. 9. Results of a dynamic vapor sorption experiment are provided in FIG. 10.

### Example 9—Preparation of Form D Crystalline Monosodium Salt of Compound I

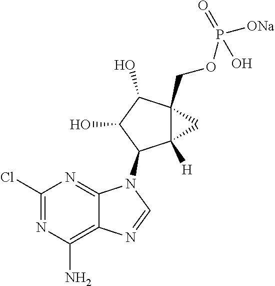

A slurry of ((1R,2R,3S,4R,5S)-4-(6-amino-2-chloro-9H-purin-9-yl)-2,3-dihydroxybicyclo[3.1.0]hexan-1-yl)methyl dihydrogen phosphate, ethanol, and sodium ethoxide was prepared and held at 50° C. for one day. Then, the title compound was isolated as a crystalline solid. An X-ray powder diffractogram of the title compound is provided in FIG. 11. Tabulated characteristics of the X-ray powder diffractogram in FIG. 11 are provided in the following table, which lists diffraction angle 2θ, inter-planar distances d, and relative intensity (expressed as a percentage with respect to the most intense peak):

### X-Ray Powder Diffractogram Data

| Angle [2θ] | d-spacing [Å] | Relative Intensity [%] |
|---|---|---|
| 5.0 | 17.8 | 14 |
| 9.2 | 9.6 | 14 |
| 9.9 | 8.9 | 18 |
| 10.4 | 8.5 | 35 |
| 13.2 | 6.7 | 14 |
| 14.2 | 6.2 | 10 |
| 15.1 | 5.8 | 17 |
| 15.7 | 5.6 | 5 |
| 16.1 | 5.5 | 9 |
| 16.8 | 5.3 | 7 |
| 17.7 | 5.0 | 20 |
| 18.4 | 4.8 | 17 |
| 18.9 | 4.7 | 80 |
| 19.2 | 4.6 | 13 |
| 19.6 | 4.5 | 15 |
| 19.9 | 4.5 | 8 |
| 20.8 | 4.3 | 9 |
| 21.5 | 4.1 | 24 |
| 21.9 | 4.0 | 13 |
| 23.6 | 3.8 | 4 |
| 24.1 | 3.7 | 20 |
| 24.9 | 3.6 | 10 |
| 26.1 | 3.4 | 8 |
| 26.6 | 3.3 | 19 |
| 26.8 | 3.3 | 100 |
| 27.8 | 3.2 | 8 |
| 28.1 | 3.2 | 38 |
| 30.0 | 3.0 | 10 |
| 31.4 | 2.8 | 13 |
| 32.4 | 2.8 | 5 |
| 33.1 | 2.7 | 7 |
| 33.8 | 2.6 | 4 |
| 38.4 | 2.3 | 8 |

Results of differential scanning calorimetry and a thermogravimetric analysis are provided in FIG. 12. Results of a dynamic vapor sorption experiment are provided in FIG. 13.

### Example 10—Preparation of Form E Crystalline Disodium Salt of Compound I

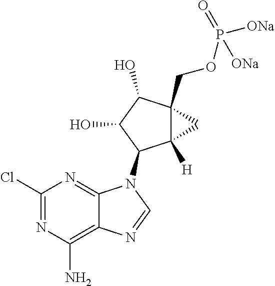

A slurry of ((1R,2R,3S,4R,5S)-4-(6-amino-2-chloro-9H-purin-9-yl)-2,3-dihydroxybicyclo[3.1.0]hexan-1-yl)methyl dihydrogen phosphate, methanol, and sodium methoxide was prepared and held at 50° C. for one day. Then, the title compound was isolated as a crystalline solid. An X-ray powder diffractogram of the title compound is provided in FIG. 14. Tabulated characteristics of the X-ray powder diffractogram in FIG. 14 are provided in the following table, which lists diffraction angle 2θ, inter-planar distances d, and relative intensity (expressed as a percentage with respect to the most intense peak):

### X-Ray Powder Diffractogram Data

| Angle [2θ] | d-spacing [Å] | Relative Intensity [%] |
|---|---|---|
| 4.6 | 19.2 | 50 |
| 9.2 | 9.6 | 17 |
| 13.8 | 6.4 | 42 |
| 15.0 | 5.9 | 14 |
| 17.6 | 5.0 | 12 |
| 18.4 | 4.8 | 34 |
| 19.2 | 4.6 | 100 |
| 20.1 | 4.4 | 3 |
| 20.9 | 4.2 | 7 |
| 21.6 | 4.1 | 7 |
| 23.4 | 3.8 | 4 |
| 24.2 | 3.7 | 7 |
| 24.9 | 3.6 | 28 |
| 25.7 | 3.5 | 4 |
| 26.5 | 3.4 | 11 |
| 27.2 | 3.3 | 24 |
| 30.9 | 2.9 | 8 |
| 32.4 | 2.8 | 9 |
| 33.4 | 2.7 | 8 |

Results of differential scanning calorimetry and a thermogravimetric analysis are provided in FIG. 15. Results of a dynamic vapor sorption experiment are provided in FIG. 16.

### Example 11—Preparation of Form F Crystalline Disodium Salt of Compound I

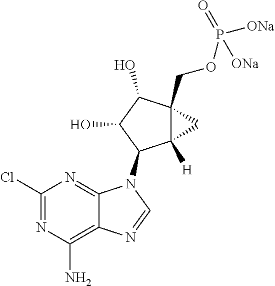

A slurry of ((1R,2R,3S,4R,5S)-4-(6-amino-2-chloro-9H-purin-9-yl)-2,3-dihydroxybicyclo[3.1.0]hexan-1-yl)methyl dihydrogen phosphate, methanol, and sodium hydroxide was prepared and held at 50° C. for one day. Then, the resulting turbid mixture was evaporated at room temperature, to provide the title compound as a crystalline solid. An X-ray powder diffractogram of the title compound is provided in FIG. 17. Tabulated characteristics of the X-ray powder diffractogram in FIG. 17 are provided in the following table, which lists diffraction angle 2θ, inter-planar distances d, and relative intensity (expressed as a percentage with respect to the most intense peak):

### X-Ray Powder Diffractogram Data

| Angle [2θ] | d-spacing [Å] | Relative Intensity [%] |
|---|---|---|
| 5.0 | 17.5 | 38 |
| 7.6 | 11.6 | 3 |
| 10.1 | 8.8 | 48 |
| 11.7 | 7.6 | 8 |
| 12.0 | 7.4 | 17 |
| 13.2 | 6.7 | 29 |
| 13.4 | 6.6 | 4 |
| 14.8 | 6.0 | 24 |
| 15.1 | 5.9 | 30 |
| 15.4 | 5.7 | 26 |
| 15.7 | 5.6 | 100 |
| 17.3 | 5.1 | 11 |
| 18.0 | 4.9 | 48 |
| 18.3 | 4.9 | 25 |
| 18.7 | 4.7 | 8 |
| 19.7 | 4.5 | 24 |
| 20.5 | 4.3 | 66 |
| 21.9 | 4.1 | 13 |
| 22.8 | 3.9 | 21 |
| 23.5 | 3.8 | 23 |
| 24.4 | 3.7 | 10 |
| 24.7 | 3.6 | 27 |
| 25.4 | 3.5 | 3 |
| 25.9 | 3.4 | 45 |
| 26.4 | 3.4 | 52 |
| 27.1 | 3.3 | 91 |
| 27.6 | 3.2 | 25 |
| 27.9 | 3.2 | 32 |
| 28.7 | 3.1 | 30 |
| 29.4 | 3.0 | 11 |
| 30.5 | 2.9 | 45 |
| 32.1 | 2.8 | 34 |
| 32.5 | 2.8 | 18 |
| 32.9 | 2.7 | 9 |
| 33.3 | 2.7 | 12 |
| 33.6 | 2.7 | 7 |
| 34.1 | 2.6 | 5 |
| 35.1 | 2.6 | 25 |
| 35.8 | 2.5 | 3 |
| 36.5 | 2.5 | 3 |
| 36.8 | 2.4 | 1 |
| 37.2 | 2.4 | 2 |
| 38.0 | 2.4 | 20 |
| 38.7 | 2.3 | 10 |

Results of differential scanning calorimetry and a thermogravimetric analysis are provided in FIG. 18. Results of a dynamic vapor sorption experiment are provided in FIG. 19.

### Example 12—Preparation of Form G Crystalline Monosodium Salt of Compound I

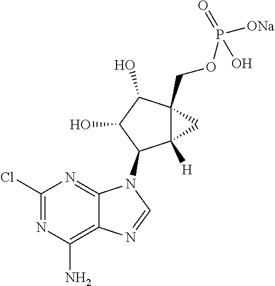

A mixture of ((1R,2R,3S,4R,5S)-4-(6-amino-2-chloro-9H-purin-9-yl)-2,3-dihydroxybicyclo[3.1.0]hexan-1-yl)methyl dihydrogen phosphate, methanol, dimethyl sulfoxide, sodium methoxide was prepared and heated to reflux for one day. Then, to the resulting turbid solution was added diethyl ether, thereby resulting in a precipitate. The mixture containing the precipate was held at room temperature for one day, then the title compound was isolated as a crystalline solid. An X-ray powder diffractogram of the title compound is provided in FIG. 20. Tabulated characteristics of the X-ray powder diffractogram in FIG. 20 are provided in the following table, which lists diffraction angle 2θ, inter-planar distances d, and relative intensity (expressed as a percentage with respect to the most intense peak):

### X-ray Powder Diffractogram Data

| Angle [2θ] | d-spacing [Å] | Relative Intensity [%] |
|---|---|---|
| 5.2 | 17.1 | 58 |
| 9.4 | 9.4 | 26 |
| 10.3 | 8.6 | 69 |
| 12.1 | 7.3 | 11 |
| 13.5 | 6.5 | 25 |
| 13.8 | 6.4 | 21 |
| 16.7 | 5.3 | 82 |
| 18.1 | 4.9 | 29 |
| 18.6 | 4.8 | 25 |
| 18.8 | 4.7 | 19 |
| 19.2 | 4.6 | 20 |
| 20.4 | 4.4 | 20 |
| 20.7 | 4.3 | 23 |
| 21.1 | 4.2 | 7 |
| 21.4 | 4.1 | 10 |
| 21.7 | 4.1 | 7 |
| 22.4 | 4.0 | 5 |
| 23.1 | 3.8 | 31 |
| 23.6 | 3.8 | 5 |
| 24.6 | 3.6 | 72 |
| 25.0 | 3.6 | 17 |
| 26.2 | 3.4 | 34 |
| 26.9 | 3.3 | 54 |
| 27.3 | 3.3 | 100 |
| 27.8 | 3.2 | 20 |
| 28.7 | 3.1 | 29 |
| 29.2 | 3.1 | 25 |
| 30.8 | 2.9 | 9 |
| 32.2 | 2.8 | 19 |
| 34.2 | 2.6 | 39 |
| 37.0 | 2.4 | 22 |

### Example 13—Preparation of Form H Crystalline Disodium Salt of Compound I

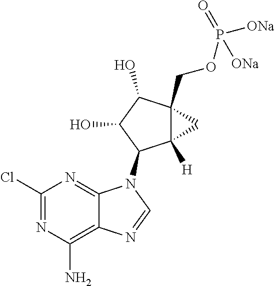

A mixture of ((1R,2R,3S,4R,5S)-4-(6-amino-2-chloro-9H-purin-9-yl)-2,3-dihydroxybicyclo[3.1.0]hexan-1-yl)methyl dihydrogen phosphate, methanol, dimethyl sulfoxide, sodium methoxide was prepared and heated to reflux. Then, the resulting mixture was cooled to room temperature, and diethyl ether was added, thereby resulting in a precipitate. The mixture containing the precipate was held at room temperature for one day. Then the resulting mixture was triturated with diethyl ether for four days. Next, the title compound was isolated and air dried at room temperature to provide the crystalline solid. An X-ray powder diffractogram of the title compound is provided in FIG. 21. Tabulated characteristics of the X-ray powder diffractogram in FIG. 21 are provided in the following table, which lists diffraction angle 2θ, inter-planar distances d, and relative intensity (expressed as a percentage with respect to the most intense peak):

### X-Ray Powder Diffractogram Data

| Angle [2θ] | d-spacing [Å] | Relative Intensity [%] |
|---|---|---|
| 5.3 | 16.6 | 20 |
| 9.4 | 9.4 | 6 |
| 10.6 | 8.3 | 39 |
| 13.2 | 6.7 | 5 |
| 14.1 | 6.3 | 27 |
| 15.3 | 5.8 | 21 |
| 16.4 | 5.4 | 5 |
| 18.5 | 4.8 | 10 |
| 19.4 | 4.6 | 21 |
| 20.2 | 4.4 | 6 |
| 21.0 | 4.2 | 21 |
| 24.4 | 3.6 | 22 |
| 26.4 | 3.4 | 98 |
| 27.1 | 3.3 | 100 |
| 33.2 | 2.7 | 22 |

### Example 14—Preparation of Form I Crystalline Disodium Salt of Compound I

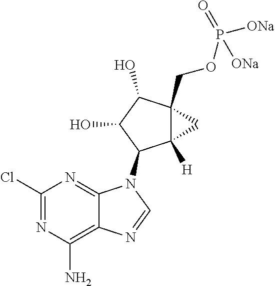

A slurry of ((1R,2R,3S,4R,5S)-4-(6-amino-2-chloro-9H-purin-9-yl)-2,3-dihydroxybicyclo[3.1.0]hexan-1-yl)methyl dihydrogen phosphate, methanol, sodium methoxide was prepared and heated to 50° C. for one day. Then, the title compound was isolated as a crystalline solid. An X-ray powder diffractogram of the title compound is provided in FIG. 22. Tabulated characteristics of the X-ray powder diffractogram in FIG. 22 are provided in the following table, which lists diffraction angle 2θ, inter-planar distances d, and relative intensity (expressed as a percentage with respect to the most intense peak):

### X-Ray Powder Diffractogram Data

| Angle [2θ] | d-spacing [Å] | Relative Intensity [%] |
|---|---|---|
| 4.6 | 19.2 | 34 |
| 9.2 | 9.6 | 15 |
| 13.1 | 6.8 | 48 |
| 14.4 | 6.2 | 36 |
| 15.6 | 5.7 | 5 |
| 17.9 | 5.0 | 62 |
| 18.4 | 4.8 | 100 |
| 18.7 | 4.7 | 35 |
| 20.6 | 4.3 | 16 |
| 22.6 | 3.9 | 10 |
| 23.3 | 3.8 | 15 |
| 24.1 | 3.7 | 30 |
| 25.7 | 3.5 | 42 |
| 26.6 | 3.3 | 9 |
| 27.0 | 3.3 | 16 |
| 27.5 | 3.2 | 51 |
| 28.5 | 3.1 | 27 |
| 29.3 | 3.0 | 27 |
| 30.2 | 3.0 | 10 |
| 31.6 | 2.8 | 7 |
| 32.8 | 2.7 | 7 |
| 34.0 | 2.6 | 9 |
| 36.2 | 2.5 | 8 |
| 37.3 | 2.4 | 5 |
| 38.7 | 2.3 | 4 |

Results of differential scanning calorimetry and a thermogravimetric analysis are provided in FIG. 23. Results of a dynamic vapor sorption experiment are provided in FIG. 24.

### Example 15—Preparation of Form J Crystalline Monosodium Salt of Compound I

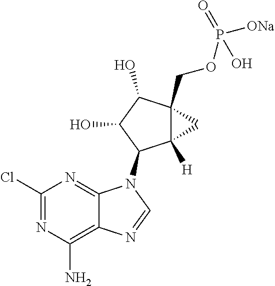

A slurry of ((1R,2R,3S,4R,5S)-4-(6-amino-2-chloro-9H-purin-9-yl)-2,3-dihydroxybicyclo[3.1.0]hexan-1-yl)methyl dihydrogen phosphate, acetone, and sodium hydroxide was prepared and held at 50° C. for one day. Then, the title compound was isolated as a crystalline solid. An X-ray powder diffractogram of the title compound is provided in FIG. 25. Tabulated characteristics of the X-ray powder diffractogram in FIG. 25 are provided in the following table, which lists diffraction angle 2θ, inter-planar distances d, and relative intensity (expressed as a percentage with respect to the most intense peak):

### X-Ray Powder Diffractogram Data

| Angle [2θ] | d-spacing [Å] | Relative Intensity [%] |
|---|---|---|
| 5.0 | 17.7 | 64 |
| 9.2 | 9.7 | 46 |
| 9.9 | 8.9 | 58 |
| 10.4 | 8.5 | 70 |
| 13.2 | 6.7 | 28 |
| 14.4 | 6.1 | 14 |
| 15.1 | 5.9 | 14 |
| 16.4 | 5.4 | 12 |
| 17.2 | 5.1 | 17 |
| 17.8 | 5.0 | 22 |
| 18.5 | 4.8 | 22 |
| 19.0 | 4.7 | 100 |
| 19.2 | 4.6 | 75 |
| 19.9 | 4.5 | 51 |
| 21.5 | 4.1 | 46 |
| 21.8 | 4.1 | 17 |
| 22.2 | 4.0 | 25 |
| 24.1 | 3.7 | 48 |
| 24.6 | 3.6 | 7 |
| 24.9 | 3.6 | 32 |
| 25.7 | 3.5 | 10 |
| 26.3 | 3.4 | 62 |
| 26.8 | 3.3 | 78 |
| 27.0 | 3.3 | 47 |
| 27.4 | 3.3 | 73 |
| 27.9 | 3.2 | 66 |
| 30.0 | 3.0 | 12 |
| 31.6 | 2.8 | 22 |
| 33.8 | 2.6 | 14 |
| 37.9 | 2.4 | 8 |
| 39.0 | 2.3 | 14 |

### Example 16—Preparation of Form N Crystalline Monosodium Salt of Compound I

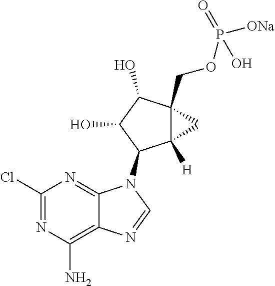

A slurry of ((1R,2R,3S,4R,5S)-4-(6-amino-2-chloro-9H-purin-9-yl)-2,3-dihydroxybicyclo[3.1.0]hexan-1-yl)methyl dihydrogen phosphate, ethanol, and sodium ethoxide was prepared and held at 50° C. for one day. Then, the title compound was isolated as a crystalline solid. An X-ray powder diffractogram of the title compound is provided in FIG. 26. Tabulated characteristics of the X-ray powder diffractogram in FIG. 26 are provided in the following table, which lists diffraction angle 2θ, inter-planar distances d, and relative intensity (expressed as a percentage with respect to the most intense peak):

### X-Ray Powder Diffractogram Data

| Angle [2θ] | d-spacing [Å] | Relative Intensity [%] |
|---|---|---|
| 4.7 | 18.7 | 31 |
| 7.6 | 11.6 | 13 |
| 9.8 | 9.0 | 37 |
| 10.2 | 8.7 | 30 |
| 11.8 | 7.5 | 10 |
| 13.3 | 6.7 | 5 |
| 13.6 | 6.5 | 6 |
| 14.0 | 6.3 | 23 |
| 15.2 | 5.8 | 30 |
| 16.1 | 5.5 | 46 |
| 16.6 | 5.3 | 5 |
| 17.7 | 5.0 | 3 |
| 18.5 | 4.8 | 100 |
| 19.6 | 4.5 | 21 |
| 20.6 | 4.3 | 4 |
| 21.0 | 4.2 | 12 |
| 21.5 | 4.1 | 27 |
| 24.1 | 3.7 | 20 |
| 24.9 | 3.6 | 18 |
| 25.6 | 3.5 | 32 |
| 26.2 | 3.4 | 58 |
| 26.7 | 3.3 | 71 |
| 27.5 | 3.2 | 35 |
| 32.0 | 2.8 | 5 |
| 32.8 | 2.7 | 41 |
| 35.1 | 2.6 | 7 |

### Example 17—Analysis of Water Solubility of Sodium Salts of Compound I

Sodium salts of Compound I described herein above were tested for solubility in water. Results are provided in the table below.

| Sodium Salt | Compound | Solvent | Aqueous Solubility |
|---|---|---|---|
| Form of | Sample | Amount | of Sodium Salt Form of |
| Compound I | Weight (mg) | (mL) | Compound I (mg/mL) |
| A | 2.3 | 0.8 | 2.9 |
| B | 3.0 | 0.7 | 4.3 |
| C | 0.3 | 0.2 | 1.5 |
| D | 3.1 | 1.3 | 2.4 |
| E | 3.3 | 1.5 | 2.2 |
| F | 2.5 | 0.7 | 3.6 |
| I | 2.7 | 0.9 | 3.0 |

### Example 18—Stability Analysis of Sodium Salt of Compound I in Aqueous Solution

Aqueous solutions of a sodium salt of Compound I were prepared and evaluated for stability. Experimental procedures and results are provided below.

### Part I—Experimental Procedure

A 100 mg aliquot of ((1R,2R,3S,4R,5S)-4-(6-amino-2-chloro-9H-purin-9-yl)-2,3-dihydroxybicyclo[3.1.0]hexan-1-yl)methyl dihydrogen phosphate was mixed with 10 mg of sodium hydroxide in an isopropyl alcohol/water (1:2 ratio by volume) solvent, to produce a clear solution (hereinafter “Solution No. 1”). A 100 mg aliquot of ((1R,2R,3S,4R,5S)-4-(6-amino-2-chloro-9H-purin-9-yl)-2,3-dihydroxybicyclo[3.1.0]hexan-1-yl)methyl dihydrogen phosphate was mixed with 10 mg of sodium hydroxide in a methanol/water (1:2 ratio by volume) solvent, to produce a clear solution (hereinafter “Solution No. 2”).

An aliquot of Solution No. 1 was heated to 50° C. and held at 50° C. for 24 hours. Purity of the solution was evaluated using high-performance liquid chromatography (HPLC) at time points 0 hours, 16 hours, and 24 hours at 50° C.

An aliquot of Solution No. 1 was heated to 70° C. and held at 70° C. for 24 hours. Purity of the solution was evaluated using HPLC at time points 0 hours, 16 hours, and 24 hours at 70° C.

An aliquot of Solution No. 2 was heated to 50° C. and held at 50° C. for 24 hours. Purity of the solution was evaluated using HPLC at time points 0 hours, 16 hours, and 24 hours at 50° C.

### Part II—Results

Results of the stability analysis according to percent purity of the solution as determined by HPLC at the indicated time points are provided in the table below.

| | | Percent Purity of Solution at | | |
|---|---|---|---|---|
| | | Indicated Time Point | | |
| Solvent System | Temperature | 0 hours | 16 hours | 24 hours |
| Solution No. 1 | 70° C. | 99.2% | 99.2% | 99.1% |
| Solution No. 1 | 50° C. | 99.2% | 99.2% | 99.2% |
| Solution No. 2 | 50° C. | 99.3% | 99.2% | 99.2% |

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A crystalline hydrate of compound I:

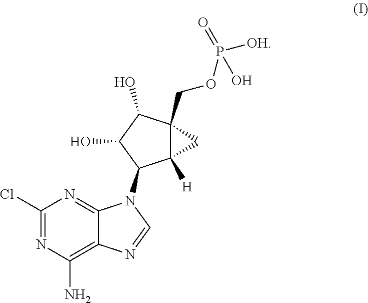 [SMILES: Nc1nc(Cl)nc2c1ncn2[C@H]1[C@H](O)[C@H](O)[C@]2(COP(=O)(O)O)C[C@H]12]

2. The crystalline hydrate compound of claim 1, wherein the crystalline form exhibits an X-ray powder diffraction pattern comprising peaks at the following diffraction angles (2θ): 8.9±0.2, 15.8±0.2, 21.3±0.2, 24.7±0.2, 26.4±0.2, 27.2±0.2, and 33.0±0.2.

3. The crystalline hydrate compound of claim 2, wherein the X-ray powder diffraction pattern further comprises a peak at the following diffraction angle (2θ): 10.1±0.2.

4. The crystalline hydrate compound of claim 3, wherein the X-ray powder diffraction pattern further comprises a peak at the following diffraction angle (2θ): 12.9±0.2.

5. The crystalline hydrate compound of claim 4, wherein the X-ray powder diffraction pattern further comprises a peak at the following diffraction angle (2θ): 18.1±0.2.

6. The crystalline hydrate compound of claim 5, wherein the X-ray powder diffraction pattern further comprises a peak at the following diffraction angle (2θ): 20.5±0.2.

7. The crystalline hydrate compound of claim 6, wherein the X-ray powder diffraction pattern further comprises a peak at the following diffraction angle (2θ): 23.8±0.2.

8. The crystalline hydrate compound of claim 7, wherein the X-ray powder diffraction pattern further comprises a peak at the following diffraction angle (2θ): 32.2±0.2.

9. The crystalline hydrate compound of claim 2, wherein the relative intensity of the peak at said diffraction angles (2θ) is at least 20%.

10. The crystalline hydrate compound of claim 2, wherein the relative intensity of the peak at said diffraction angles (2θ) is at least 15%.

11. The crystalline hydrate compound of claim 1 characterized by the following X-ray powder diffraction pattern expressed in terms of diffraction angle 2θ, inter-planar distances d, and relative intensity (expressed as a percentage with respect to the most intense peak):

| Angle [2θ] | d-spacing [Å] | Relative Intensity [%] |
|---|---|---|
| 5.1 | 17.3 | 30 |
| 8.9 | 9.9 | 66 |
| 9.8 | 9.0 | 28 |
| 10.1 | 8.7 | 49 |
| 12.9 | 6.8 | 15 |
| 14.7 | 6.0 | 54 |
| 15.1 | 5.8 | 2 |
| 15.8 | 5.6 | 21 |
| 16.4 | 5.4 | 8 |
| 17.0 | 5.2 | 18 |
| 18.1 | 4.9 | 30 |
| 18.4 | 4.8 | 22 |
| 18.8 | 4.7 | 48 |
| 19.2 | 4.6 | 16 |
| 19.4 | 4.6 | 21 |
| 19.7 | 4.5 | 30 |
| 20.5 | 4.3 | 14 |
| 21.3 | 4.2 | 58 |
| 22.1 | 4.0 | 13 |
| 22.4 | 4.0 | 11 |
| 22.9 | 3.9 | 11 |
| 23.8 | 3.7 | 15 |
| 24.7 | 3.6 | 58 |
| 25.4 | 3.5 | 14 |
| 26.0 | 3.4 | 13 |
| 26.4 | 3.4 | 100 |
| 27.2 | 3.3 | 41 |
| 27.8 | 3.2 | 21 |
| 30.5 | 2.9 | 7 |
| 30.9 | 2.9 | 7 |
| 31.8 | 2.8 | 8 |
| 32.2 | 2.8 | 20 |
| 33.0 | 2.7 | 24 |
| 33.5 | 2.7 | 14 |
| 36.6 | 2.5 | 7 |
| 37.7 | 2.4 | 13 |
| 38.9 | 2.3 | 21 |

12. The crystalline hydrate compound of claim 1, wherein the compound has an X-ray powder diffraction pattern substantially as shown in FIG. 4.

13. The crystalline hydrate compound of claim 2, wherein the mole ratio of water to compound I is from 1.4:1 to 1.5:1.

14. The crystalline hydrate compound of claim 2, wherein the mole ratio of water to compound I is about 1.5:1.

15. The crystalline hydrate compound of claim 1, wherein the crystalline form exhibits an X-ray powder diffraction pattern comprising peaks at the following diffraction angles (2θ): 10.3±0.2, 13.8±0.2, 19.0±0.2, 20.7±0.2, 21.8±0.2, 23.5±0.2, and 27.3±0.2.

16-23. (canceled)

24. The crystalline hydrate compound of claim 15 characterized by the following X-ray powder diffraction pattern expressed in terms of diffraction angle 2θ, inter-planar distances d, and relative intensity (expressed as a percentage with respect to the most intense peak):

| Angle [2θ] | d-spacing [Å] | Relative Intensity [%] |
|---|---|---|
| 5.2 | 17.1 | 18 |
| 6.5 | 13.6 | 18 |
| 9.5 | 9.3 | 26 |
| 10.3 | 8.6 | 52 |
| 11.2 | 7.9 | 7 |
| 12.5 | 7.1 | 8 |
| 13.1 | 6.8 | 6 |
| 13.8 | 6.4 | 73 |
| 13.9 | 6.4 | 24 |
| 15.4 | 5.7 | 14 |
| 16.2 | 5.5 | 21 |
| 16.9 | 5.2 | 10 |
| 18.1 | 4.9 | 26 |
| 18.7 | 4.7 | 41 |
| 19.0 | 4.7 | 78 |
| 19.6 | 4.5 | 40 |
| 20.7 | 4.3 | 53 |
| 21.8 | 4.1 | 54 |
| 22.7 | 3.9 | 11 |
| 23.2 | 3.8 | 14 |
| 23.5 | 3.8 | 45 |
| 24.7 | 3.6 | 11 |
| 24.9 | 3.6 | 11 |
| 26.0 | 3.4 | 9 |
| 26.2 | 3.4 | 16 |
| 26.7 | 3.3 | 13 |
| 27.3 | 3.3 | 100 |
| 28.3 | 3.1 | 22 |
| 28.7 | 3.1 | 13 |
| 31.0 | 2.9 | 13 |
| 32.9 | 2.7 | 28 |
| 33.4 | 2.7 | 11 |
| 34.6 | 2.6 | 5 |
| 35.3 | 2.5 | 4 |
| 35.6 | 2.5 | 14 |
| 37.4 | 2.4 | 15 |
| 37.7 | 2.4 | 10 |
| 38.2 | 2.4 | 10 |
| 38.5 | 2.3 | 5 |
| 39.2 | 2.3 | 3 |

25-28. (canceled)

29. The crystalline hydrate compound of claim 15, wherein the mole ratio of water to compound I is about 1:1.

30-44. (canceled)

45. A pharmaceutical composition comprising a compound of claim 1 and a pharmaceutically acceptable carrier.

46. A method for treating a cardiac disorder, comprising administering to a subject in need thereof a therapeutically effective amount of a pharmaceutical composition of claim 45 to treat the cardiac disorder.

47. A method for improving cardiac contractile performance in a subject, comprising administering to a subject in need thereof an effective amount of a pharmaceutical composition of claim 45 to improve cardiac contractile performance.

48-52. (canceled)

53. The method of claim 46, wherein the cardiac disorder is heart failure.

54-55. (canceled)

56. A method of preparing a crystalline hydrate of compound I, comprising the steps of:
(a) admixing compound I, methanol, and water to form a mixture, and heating said mixture to a temperature greater than 30° C. to form a heated mixture; and
(b) cooling said heated mixture to thereby form a precipitate that is the crystalline hydrate of compound I;
wherein compound I is represented by:

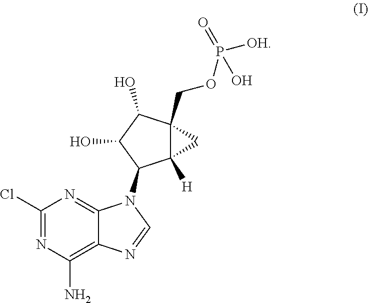 [SMILES: Nc1nc(Cl)nc2c1ncn2[C@H]1[C@H](O)[C@H](O)[C@]2(COP(=O)(O)O)C[C@H]12]

57-61. (canceled)

62. A method of preparing a pharmaceutical composition, comprising admixing (i) a crystalline hydrate compound of claim 1 and (ii) a pharmaceutically acceptable carrier.

63. (canceled)

64. The method of claim 62, wherein the method comprises admixing (i) a crystalline hydrate compound of claim 2 and (ii) a pharmaceutically acceptable carrier.

65. (canceled)

66. A pharmaceutical composition made according to a method of claim 64.
